(19) United States
(12) Patent Application Publication
(10) Pub. No.: US 2001/0041353 A1
(43) Pub. Date: Nov. 15, 2001

(54) Novel SSP-1 compositions and therapeutic and diagnostic uses therefor

(75) Inventor: Sean Anthony McCarthy, Boston, MA (US)

Correspondence Address: Patent Group, Foley, Hoag & Eliot, LLP, One Post Office Square, Boston, MA, 02109-2170, US

(73) Assignee: Millennium Biotherapeutics

(21) Appl. No.: 09/755,958

(22) Filed: Jan. 5, 2001

Related U.S. Application Data
(63) Continuation of application No. 08/843,656, filed on Apr. 16, 1997.

(51) Int. Cl. C12Q 1/68; A61K 38/43; C12N 9/16; C12P 21/02; C12N 5/06; C07H 21/04

(52) U.S. Cl. 435/69.1; 435/196; 435/325; 435/6; 424/94.1; 536/23.2

(57) ABSTRACT

The present invention relates to the discovery of novel genes encoding Small Secreted Protein-1 (SSP-1) polypeptides. Therapeutics, diagnostics and screening assays based on these molecules are also disclosed.

### BACKGROUND OF THE INVENTION

Multicellular organisms have an elaborate cell-to-cell communication network, coordinating growth, differentiation and metabolism of the multitude of cells in the diverse tissues and organs. Communication may be direct cell-to-cell contact and establishment of specific contacts between cells is often a necessary step in cell differentiation.

Cells may also communicate over longer distances, and in such cases, extracellular products act as signals. These products are synthesized and released by signaling cells and then move to other cells where they induce a specific response in those target cells having receptors for the signaling cells. In plants and animals, extracellular signals control the growth of most tissues, govern the synthesis and secretion of proteins, and regulate the composition of intracellular and extracellular fluids.

Secreted proteins play an integral role in the formation, differentiation, and maintenance of cells in multicellular organisms. Hence identification of genes encoding secreted proteins is of significant importance in understanding fundamental biological processes and for modulating such processes. It is known in the art that a vast array of different proteins are secreted by different vertebrate cells and many cells are specialized for the secretion of specific proteins. These secretory proteins may be roughly divided into the following classes: serum proteins, extracellular matrix proteins, peptide hormones, enzymes, growth factors, lymphokines etc. After synthesis, secretory proteins are localized to the lumen of the rough endoplasmic reticulum. These proteins are always surrounded by membrane bound vesicles and they migrate to the surface of the Golgi vesicles, where they undergo modifications and then migrate to the cell surface where they undergo further modifications. The proteins are shuttled between the Golgi vesicles by small transport vesicles. In certain cells, the secretory proteins, such as the serum proteins, are continuously synthesized and secreted. In other cells, the secretion is not continuous, and the proteins may be stored in the secretory vesicles and await a signal for secretion.

Of particular importance in the development and maintenance of tissue in vertebrate animals is a type of extracellular communication called induction, which occurs between neighboring cell layers and tissues (Saxen et al. (1989) Int J Dev Biol 33:21-48; and Gurdon et al. (1987) Development 99:285-306). In inductive interactions, chemical signals secreted by one cell population influence the developmental fate of a second cell population. Typically, cells responding to the inductive signals are diverted from one cell fate to another, neither of which is the same as the fate of the signaling cells.

### SUMMARY OF THE INVENTION

The present invention is based, at least in part, on the discovery of a gene encoding a secreted human protein, referred to herein as “SSP-1”. An exemplary SSP-1 molecule has been deposited with the American Type Culture Collection (ATCC) on ______and has been assigned ATCC designation number ______. The human SSP-1 gene transcript is shown in FIG. 1 (SEQ ID NO 1) and includes 5′ and 3′ untranslated regions and a 516 base pair open reading frame (SEQ ID NO 3) encoding a 172 amino acid polypeptide having SEQ ID NO. 2. The mature protein (i.e. secreted protein minus the signal sequence) is comprised of about 149 amino acids.

An amino acid and nucleotide sequence analysis using the BLAST program (Altschul et al. (1990) J. Mol. Biol. 215:403) revealed that the amino acid and nucleic acid sequences of the newly identified human SSP-1 protein and gene show no significant sequence similarity to any protein and DNA sequence in the databases.

The human SSP-1 protein has significant amino acid sequence similarity to two Xenopus Laevis proteins, which are expressed during the development of the cement gland in Xenopus embryos. However, in mammals there is no known equivalent organ to the cement gland.

Based at least in part on the fact that SSP-1 is a secreted protein having a small molecular size, SSP-1 is likely to be a cellular communication factor, such as a cytokine. Accordingly, SSP-1 is likely to regulate cell proliferation, differentiation and/or survival. Alternatively, SSP-1 could also interact with an extracellular molecule, e.g., a protein, and be involved in the transport of such molecule, or SSP-1 can be a structural protein.

In one aspect, the invention features isolated SSP-1 nucleic acid molecules. In one embodiment, the SSP-1 nucleic acid is from a vertebrate. In a preferred embodiment, the SSP-1 nucleic acid is from a mammal, e.g. a human. In an even more preferred embodiment, the nucleic acid has the nucleic acid sequence set forth in SEQ ID NO. 1 and/or 3 or a portion thereof. The disclosed molecules can be non-coding, (e.g. a probe, antisense, or ribozyme molecules) or can encode a functional SSP-1 polypeptide (e.g. a polypeptide which specifically modulates biological activity, by acting as either an agonist or antagonist of at least one bioactivity of the human SSP-1 polypeptide). In one embodiment, the nucleic acid molecules can hybridize to the SSP-1 gene contained in ATCC designation number ______ or to the complement of the SSP-1 gene contained in ATCC designation number . In another embodiment, the nucleic acids of the present invention can hybridize to a vertebrate SSP-1 gene or to the complement of a vertebrate SSP-1 gene. In a further embodiment, the claimed nucleic acid can hybridize with a nucleic acid sequence shown in FIG. 1 (SEQ ID NOS. 1 and 3). In a preferred embodiment, the hybridization is conducted under mildly stringent or stringent conditions.

In further embodiments, the nucleic acid molecule is a SSP-1 nucleic acid that is at least about 70%, preferably about 80%, more preferably about 85%, and even more preferably at least about 90% or 95% homologous to the nucleic acid shown as SEQ ID NOS: 1 or 3 or to the complement of the nucleic acid shown as SEQ ID NOS: 1 or 3. In a further embodiment, the nucleic acid molecule is a SSP-1 nucleic acid that is at least about 70%, preferably at least about 80%, more preferably at least about 85% and even more preferably at least about 90% or 95% similar in sequence to the SSP-1 nucleic acid contained in ATCC designation number ______ or to the complement of the SSP-1 nucleic acid contained in ATCC designation number ______.

The invention also provides probes and primers comprising substantially purified oligonucleotides, which correspond to a region of nucleotide sequence which hybridizes to at least about 6 at least about 10, and at least about 15, at least about 20, or preferably at least about 25 consecutive nucleotides of the sequence set forth as SEQ ID NO. 1 or complements of the sequence set forth as SEQ ID NO. 1 or naturally occurring mutants thereof. In preferred embodiments, the probe/primer further includes a label group attached thereto, which is capable of being detected.

For expression, the subject nucleic acids can include a transcriptional regulatory sequence, e.g. at least one of a transcriptional promoter (e.g., for constitutive expression or inducible expression) or transcriptional enhancer sequence, which regulatory sequence is operably linked to the gene sequence. Such regulatory sequences in conjunction with a SSP-1 nucleic acid molecule can provide a useful vector for gene expression. This invention also describes host cells transfected with said expression vector whether prokaryotic or eukaryotic and in vitro (e.g. cell culture) and in vivo (e.g. transgenic) methods for producing SSP-1 proteins by employing said expression vectors.

In another aspect, the invention features the isolated SSP-1 polypeptide, preferably substantially pure preparations, e.g. of plasma purified or recombinantly produced polypeptide. In particularly preferred embodiments, the subject polypeptide has a SSP-1 bioactivity, for example, it is capable of modulating cell proliferation, differentiation and/or survival.

In a preferred embodiment, the polypeptide is encoded by a nucleic acid which hybridizes with the nucleic acid sequence represented in SEQ ID NOS. 1 and 3. In a further preferred embodiment, the SSP-1 polypeptide is comprised of the amino acid sequence set forth in SEQ ID No. 2. The subject SSP-1 protein also includes within its scope modified proteins, e.g. proteins which are resistant to post-translational modification, for example, due to mutations which alter modification sites (such as tyrosine, threonine, serine or aspargine residues), or which prevent glycosylation of the protein, or which prevent interaction of the protein with intracellular proteins involved in signal transduction.

The SSP-1 polypeptides of the present invention can be glycosylated, or conversely, by choice of the expression system or by modification of the protein sequence to preclude glycosylation, reduced carbohydrate analogs can also be provided. Glycosylated forms can be obtained based on derivatization with glycosaminoglycan chains. Also, SSP-1 polypeptides can be generated which lack an endogenous signal sequence (though this is typically cleaved off even if present in the pro-form of the protein).

The products of the SSP-1 gene are likely to be involved in the formation and maintenance of ordered spatial arrangements of differentiated tissues in vertebrates, both adult and embryonic, and can be used to generate and/or maintain an array of different vertebrate tissue both in vitro and in vivo.

In general, the invention features SSP-1 polypeptides, preferably substantially pure preparations of one or more of the subject SSP-1 polypeptides. The invention also provides recombinantly produced SSP-1 polypeptides. In preferred embodiments the polypeptide has a biological activity including: an ability to modulate proliferation, differentiation and/or survival of tissue.

The SSP-1 polypeptide can comprise a full length protein or can comprise smaller fragments corresponding to one or more particular motifs/domains, or fragments comprising at least about 5, 10, 25, 50, 75, 100, 125, 130, 135, 140 or 145 amino acids in length. In preferred embodiments, the polypeptide has an SSP-1 bioactivity, such as the capability to modulate cell proliferation, differentiation and/or survival.

In yet another preferred embodiment, the invention features a purified or recombinant polypeptide, which has the ability to modulate, e.g., mimic or antagonize, an activity of a wild-type SSP-1 protein. Preferably, the polypeptide comprises an amino acid sequence identical or homologous to a sequence designated in SEQ ID No: 2.

Another aspect of the invention features chimeric molecules (e.g., fusion proteins) comprising a SSP-1 protein. For instance, the SSP-1 protein can be provided as a recombinant fusion protein which includes a second polypeptide portion, e.g., a second polypeptide having an amino acid sequence unrelated (heterologous) to the SSP-1 polypeptide.

Yet another aspect of the present invention concerns an immunogen comprising a SSP-1 polypeptide in an immunogenic preparation, the immunogen being capable of eliciting an immune response specific for a SSP-1 polypeptide; e.g. a humoral response, an antibody response and/or cellular response. In a preferred embodiment, the immunogen comprises an antigenic determinant, e.g. a unique determinant of a protein encoded by the nucleic acid set forth in SEQ ID No. 1 or 3; or as set forth in SEQ ID NO.2.

A still further aspect of the present invention features antibodies and antibody preparations specifically reactive with an epitope of a SSP-1 protein.

The invention also features transgenic non-human animals which include (and preferably express) a heterologous form of a SSP-1 gene described herein, or which misexpress an endogenous SSP-1 gene (e.g., an animal in which expression of one or more of the subject SSP-1 proteins is disrupted). Such transgenic animals can serve as animal models for studying cellular and/or tissue disorders comprising mutated or misexpressed SSP-1 alleles or for use in drug screening. Alternatively, such transgenic animals can be useful for expressing recombinant SSP-1 polypeptides.

A further aspect of the present invention provides methods for determining whether a subject is at risk for a disorder characterized by an aberrant (e.g., too high, too low) SSP-1 activity such as an aberrant-SSP-1 expression. Accordingly, in one embodiment, the method includes detecting, in a tissue of the subject, the presence or absence of a genetic lesion characterized by at least one of the following: (i) a mutation of a gene encoding a SSP-1 protein, e.g. represented in SEQ ID No: 1 or a homolog thereof; (ii) the mis-expression of a SSP-1 gene or (iii) an error or mutation in the promoter that may lead to aberrant expression. In preferred embodiments, detecting the genetic lesion includes ascertaining the existence of at least one of: a deletion of one or more nucleotides from a gene; an addition of one or more nucleotides to the gene; a substitution of one or more nucleotides of the gene; a gross chromosomal rearrangement of the gene; an alteration in the level of a messenger RNA transcript of the gene; the presence of a non-wild type splicing pattern of a messenger RNA transcript of the gene; and/or a non-wild type level of the SSP-1 protein.

For example, detecting a genetic lesion can include (i) providing a probe/primer comprised of an oligonucleotide which hybridizes to a sense or antisense sequence of a SSP-1 gene or naturally occurring mutants thereof, or 5′ or 3′ flanking sequences naturally associated with the SSP-1 gene; (ii) contacting the probe/primer to an appropriate nucleic acid containing sample; and (iii) detecting, by hybridization of the probe/primer to the nucleic acid, the presence or absence of the genetic lesion; e.g. wherein detecting the lesion comprises utilizing the probe/primer to determine the nucleotide sequence of the SSP-1 gene and, optionally, of the flanking nucleic acid sequences. For instance, the primer can be employed in a polymerase chain reaction (PCR) or in a ligation chain reaction (LCR). In alternate embodiments, the level of a SSP-1 protein is detected in an immunoassay using an antibody which is specifically immunoreactive with a wild-type or mutated SSP-1 protein.

An exemplary method for identifying a compound which modulates an SSP-1 activity includes the steps of (a) forming a reaction mixture including: (i) a SSP-1 polypeptide, (ii) an SSP-1 binding partner (e.g. receptor either related or present on a cell surface), and (iii) a test compound; and (b) detecting interaction of the SSP-1 and the SSP-1 binding protein. A statistically significant change (potentiation or inhibition) in the interaction of the SSP-1 and SSP-1 binding protein in the presence of the test compound, relative to the interaction in the absence of the test compound, indicates a potential agonist (mimetic or potentiator) or antagonist (inhibitor) of SSP-1 bioactivity for the test compound. The reaction mixture can be a cell-free protein preparation, e.g., a reconstituted protein mixture or a cell lysate, or it can be a recombinant cell including a heterologous nucleic acid recombinantly expressing the SSP-1 binding partner.

In preferred embodiments, the step of detecting interaction of the SSP-1 and SSP-1 binding partner (e.g. receptor) is a competitive binding assay.

In preferred embodiments, at least one of the SSP-1 polypeptide and the SSP-1 binding partner comprises a detectable label, and interaction of the SSP-1 and SSP-1 binding partner is quantified by detecting the label in the complex. The detectable label can be, e.g., a radioisotope, a fluorescent compound, an enzyme, or an enzyme cofactor. In other embodiments, the complex is detected by an immunoassay.

Yet another exemplary embodiment provides an assay for screening test compounds to identity agents which modulate the binding of SSP-1 proteins with a SSP-1 receptor, comprising: (i) providing a cell expressing a SSP-1 receptor; (ii) contacting the cell with a SSP-1 polypeptide and a test compound; and (iii) detecting interaction of the SSP-1 polypeptide and receptor. A statistically significant change in the level of interaction of the SSP-1 polypeptide and receptor is indicative of an agent that modulates the interaction of SSP-1 proteins with a SSP-1 receptor. The interaction of the SSP-1 polypeptide and receptor can be detected. e.g., by detecting change in phenotype of the cell relative to the absence of the test compound. The change in phenotype may be, to illustrate, a gain or loss of expression of a cell-type specific marker.

In still other embodiments, the receptor transduces a signal in the cell which is sensitive to SSP-1 binding, and interaction of the SSP-1 polypeptide and receptor are detected by detecting change in the level of an intracellular second messenger responsive to signaling by the receptor. For example, interaction of the SSP-1 polypeptide and receptor can be detected by changes in intracellular protein phosphorylation.

In other embodiments, the receptor transduces a signal in the cell which is sensitive to SSP-1 binding, and the cell further comprises a reporter gene construct comprising a reporter gene in operable linkage with a transcriptional regulatory sequence sensitive to intracellular signals transduced by interaction of the SSP-1 polypeptide and receptor, expression of the reporter gene providing a detectable signal for detecting interaction of the SSP-1 polypeptide and receptor. The reporter gene can encode, e.g., a gene product that gives rise to a detectable signal such as: color, fluorescence, luminescence, cell viability relief of a cell nutritional requirement, cell growth, and drug resistance. For example, the reporter gene can encode a gene product selected from the group consisting of chloramphenicol acetyl transferase, luciferase, beta-galactosidase and alkaline phosphatase.

Other features and advantages of the invention will be apparent from the following detailed description and claims.

### BRIEF DESCRIPTION OF THE FIGURES

FIG. 1 shows the nucleotide sequence of the human SSP-1 gene including 5′ and 3′ untranslated regions and coding sequences (SEQ ID NO. 1)and the deduced amino acid sequence of the SSP-1 protein (SEQ ID NO 2).

FIG. 2 shows a hydrophobicity profile of the human SSP-1 protein having SEQ ID NO. 2, indicating the presence of a hydrophobic region in the NH2 terminus of the protein.

FIG. 3 show an amino acid sequence alignment of SSP-1 with two Xenopus proteins: XAG having GenBank Accession No. U76752 (Sive et al., (1989) Cell 58:171-180; Sive and Bradley (1996) Dev. Dyn. 205:265); and np77 having GenBank Accession No. U82110.

### DETAILED DESCRIPTION OF THE INVENTION

General

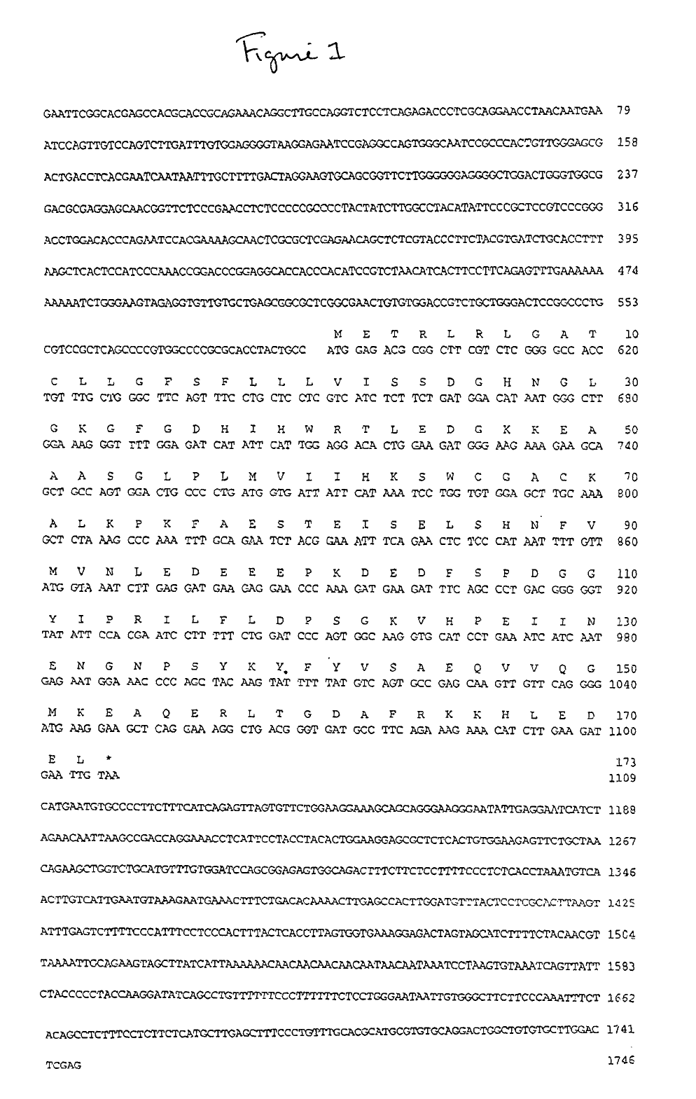

The invention is based at least in part on the discovery of a human gene encoding a secreted protein, referred to herein as Small Secreted Protein 1 (SSP-1). A full length cDNA encoding SSP-1 was cloned from a human fetal brain cDNA library. The nucleic acid sequence encoding the full length SSP-1 protein is shown in FIG. 1 and is set forth as SEQ ID NO. 1. The full length protein encoded by this nucleic acid is comprised of about 172 amino acids and has the amino acid sequence shown in FIG. 1 and set forth as SEQ ID No. 2. The coding portion (open reading frame) of SEQ ID No. I is set forth as SEQ ID No. 3 and corresponds to nucleotides 591 to 1106 of SEQ ID No. 1.

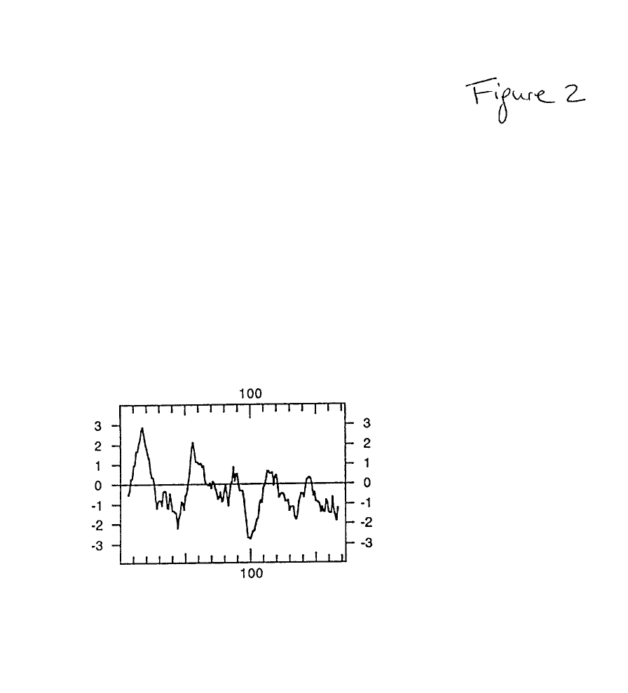

Determination of the hydrophobicity profile of human SSP-1 having the amino acid sequence set forth in SEQ ID NO.2 indicated the presence of a hydrophobic region from about amino acid 1 to about amino acid 23 of SEQ ID NO.2 (FIG. 2). Further analysis of the amino acid sequence SEQ ID NO.2 using signal peptide prediction programs predicts the presence of a signal peptide from about amino acid 1 to about amino acid 23 of SEQ ID NO.2. Accordingly, the mature SSP-1 protein is comprised of about 149 amino acids spanning from about amino acid 24 to about amino acid 172 of SEQ ID NO.2. The presence of the signal sequence, in addition to the fact that SSP-1 has been identified using a signal sequence trap system confirms that SSP-1 is a secreted protein.

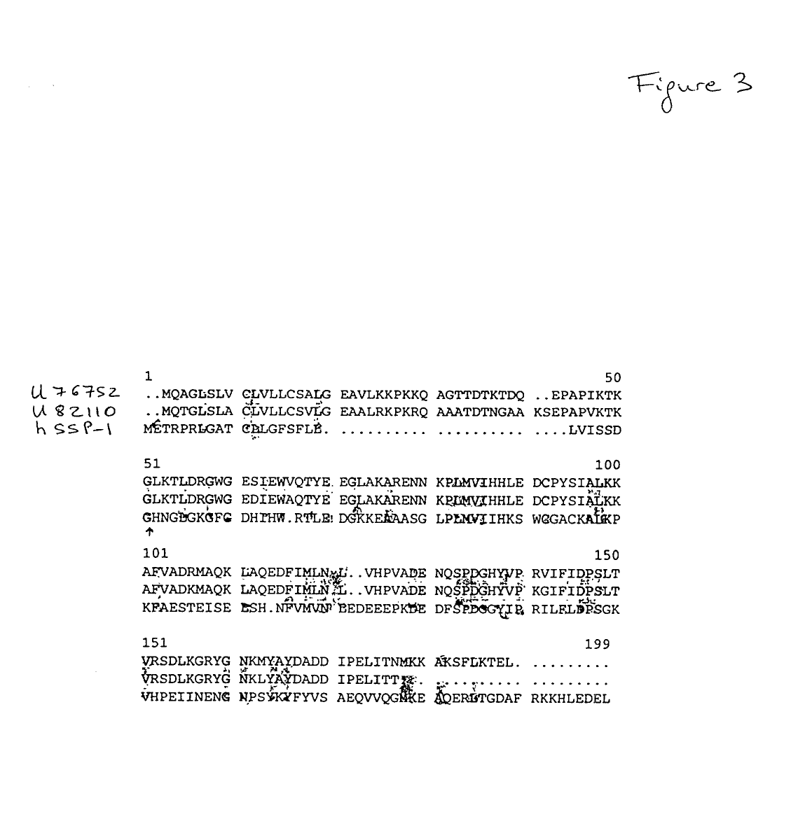

ABLAST search (Altschul et al. (1990) J. Mol. Biol. 215:403) of the nucleic acid and the amino acid sequences of SSP-1 has revealed that SSP-1 has significant homology to two proteins, which are homologous to each other, and which are expressed during the development of the cement gland in Xenopus. One protein is a 183 amino acid protein termed XAG having GenBank Accession No. U76752 (Sive et al., (1989) Cell 58:171-180; Sive and Bradley (1996) Dev. Dyn. 205:265). The second protein is a 173 amino acid protein termed np77 having GenBank Accession No, U82110. An alignment of the amino acid sequences of the two Xenopus cement gland proteins and human SSP-1 having SEQ ID NO. 2 is shown in FIG. 3. This sequence alignment shows an overall homology between the three proteins. More specifically, the amino acid sequence of the human SSP-1 having SEQ ID No. 2 is about 29.7% identical to the amino acid sequence of XAG. The amino acid sequence of human SSP-1 having SEQ ID No. 2 is about 29.4% identical to the amino acid sequence of np77. Furthermore, specific amino acid sequences are present in the three sequences, thus indicating that these sequences encode specific domains.

The cement gland is an ectodermal mucus-secreting organ, also termed “adhesive organ” located in the head of frog embryos. It produces a waterproof glue that attaches the newly hatched embryo to a solid support when it cannot yet swim efficiently and cannot feed.

Due to its small size, SSP-1 is likely to be a protein involved in communication between cells, e.g., a cytokine or chemokine and is, accordingly, likely to be involved in regulating cell proliferation, differentiation and/or cell death or cell migration. SSP-1 can also be a protein interacting with other extracellular proteins and having, e.g., a structural function or a defense function. SSP-1 may also be the first identified member of a family of small secreted proteins, which regulate cell proliferation, differentiation and/or cell death, some members of which could be cell membrane proteins.

Accordingly, the invention provides nucleic acids encoding SSP-1 proteins, fragments thereof and homologs or variants thereof The invention also provides SSP-1 polypeptides, fragments thereof and homologs or variants thereof

Since SSP-1 is likely to be involved in regulating cell proliferation, differentiation, and/or cell death, a mutated form of SSP-1, resulting in an aberrant SSP-1 activity, is likely to cause or contribute to diseases, conditions or disorders characterized by abnormal cell proliferation and/or differentiation or survival. SSP-1 could also be involved in diseases, conditions, or disorders characterized by an abnormal extracellular structure or an abnormality in a defense mechanism. Accordingly, the invention provides methods for determining whether a subject is at risk of developing or has developed a disease associated with an aberrant SSP-1 activity. Such assays can, for example, consist of determining whether the subject has a genetic lesion in an SSP-1 gene.

The invention further provides methods for treating or preventing diseases caused by or contributed to by an aberrant SSP-1 activity and/or by abnormal cell proliferation, differentiation or survival. Also within the scope of the invention are methods for identifying SSP-1 therapeutics, i.e., compounds, which are either SSP-1 agonists or SSP-1 antagonists.

Accordingly, certain aspects of the present invention relate to nucleic acid molecules encoding SSP-1 proteins, antisense molecules, ribozymes and triplex molecules that block expression of SSP-1 genes, SSP-1 proteins, antibodies immunoreactive with SSP-1 proteins, and preparations of such immunogenic compositions. In addition, the present invention relates to therapies, which are based on upmodulating (e.g., stimulating) or downmodulating (e.g., inhibiting or suppressing) SSP-1 genes and proteins. Moreover, the present invention provides diagnostic assays and reagents for detecting and treating disorders involving, for example, aberrant expression (or loss thereof) of SSP-1 genes. Other aspects of the invention are described below or will be apparent to those skilled in the art in light of the present disclosure.

Definitions

For convenience, the meaning of certain terms and phrases employed in the specification, examples, and appended claims are provided below.

The term “agonist”, as used herein, is meant to refer to an agent that mimics or upregulates (e.g. potentiates or supplements) a SSP-1 bioactivity. An SSP-1 agonist can be a compound which mimics a bioactivity of an SSP-1 protein, such as transduction of a signal from an SSP-1 receptor, by, e.g., interacting with an SSP-1 receptor. A SSP-1 agonist can also be a compound that upregulates expression of a SSP-1 gene. An SSP-1 agonist can also be a compound which modulates the expression or activity of a protein which is located downstream of an SSP-1 receptor, thereby mimicking or enhancing the effect of binding of SSP-1 to an SSP-1 receptor.

“Antagonist” as used herein is meant to refer to an agent that inhibits, decreases or suppresses an SSP-1 bioactivity. An antagonist can be a compound which decreases signalling from a SSP-1 protein, e.g., a compound that is capable of binding to SSP-1 or to an SSP-1 receptor. A preferred SSP-1 antagonist inhibits the interaction between a SSP-1 protein and another molecule, such as an SSP-1 receptor. Alternatively, a SSP-1 antagonist can be a compound that downregulates expression of a SSP-1 gene. A SSP-1 antagonist can also be a compound which modulates the expression or activity of a protein which is located downstream of an SSP-1 receptor, thereby antagonizing the effect of binding of SSP-1 to an SSP-1 receptor.

“Biological activity” or “bioactivity” or “activity” or “biological function”, which are used interchangeably, for the purposes herein means an effector or antigenic function that is directly or indirectly performed by a SSP-1 polypeptide (whether in its native or denatured conformation), or by any subsequence thereof Biological activities include binding to a second molecule, e.g., a protein, such as an SSP-1 receptor; transduction of an intracellular signal from an SSP-1 receptor; regulation of expression of genes whose expression is modulated by binding of SSP-1 to a receptor; induction of cellular proliferation; induction of cellular differentiation; modulation of cell death, such as stimulation of cell survival; and/or immune modulation, whether presently known or inherent. An SSP-1 activity can also be an enzymatic activity or a detoxifying activity, such as by binding to and eliminating an undesirable molecule. A SSP-1 bioactivity can be modulated by affecting directly a SSP-1 protein. Alternatively, a SSP-1 bioactivity can be modulated by modulating the level of a SSP-1 protein, such as by modulating expression of a SSP-1 gene. Antigenic functions include possession of an epitope or antigenic site that is capable of cross-reacting with antibodies raised against a naturally occurring or denatured SSP-1 polypeptide or fragment thereof.

Biologically active SSP-1 polypeptides include polypeptides having both an effector and antigenic function, or only one of such functions. SSP- l includes antagonist polypeptides and native SSP-1, provided that such antagonists include an epitope of a native SSP-1. An effector function of SSP-1 can be any of the above described biological activities..

As used herein the term “bioactive fragment of a SSP-1 protein” refers to a fragment of a full-length SSP-1 protein, wherein the fragment specifically mimics or antagonizes the activity of a wild-type SSP-1 protein. The bioactive fragment preferably is a fragment capable of binding to a second protein, e.g., a receptor.

The term “an aberrant activity”, as applied to an activity of a protein such as SSP-1, refers to an activity which differs from the activity of the wild-type or native protein or which differs from the activity of the protein in a healthy subject. An activity of a protein can be aberrant because it is stronger than the activity of a wildtype in a healthy subject. Alternatively, an activity can be aberrant because it is weaker or absent related to the activity of its native counterpart. An aberrant activity can also be a change in an activity. For example an aberrant protein can interact with a different protein relative to its native counterpart. A cell can have an aberrant SSP-1 activity due to overexpression or underexpression of the gene encoding SSP-1.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Appropriate hosts include, but are not limited to: bacterial cells, such as E. Coli, Salmonella typhirium; fungal cells, such as yeast; animal cells such as CH0, C127, 3T3, BHk and COS-7 cell lines.

A “chimeric protein” or “fusion protein” is a fusion of a first amino acid sequence encoding the subject SSP-1 polypeptide with a second amino acid sequence defining a domain (e.g., polypeptide portion) foreign to and not substantially homologous with any domain of one of the SSP-1 polypeptides. A chimeric protein may present a foreign domain which is found (albeit in a different protein) in an organism which also expresses the first protein, or it may be an “interspecies”, “intergenic”, etc. fusion of protein structures expressed by different kinds of organisms. In general, a fusion protein can be represented by the general formula X—SSP-1 —Y, wherein SSP-1 represents a portion of the protein which is derived from one of the SSP-1 protein, and X and Y are independently absent or represent amino acid sequences which are not related to one of the SSP-1 amino acid sequences in an organism, including naturally occurring mutants.

“Complementary” sequences as used herein refer to sequences which have sufficient complementarity to be able to hybridize, forming a stable duplex.

A “delivery complex” shall mean a targeting means (e.g., a molecule that results in higher affinity binding of a gene, protein, polypeptide or peptide to a target cell surface and/or increased cellular uptake by a target cell). Examples of targeting means include: sterols (e.g., cholesterol), lipids (e.g., a cationic lipid, virosome or liposome), viruses (e.g., adenovirus, adeno-associated virus, and retrovirus) or target cell specific binding agents (e.g., ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

As is well known, genes for a particular polypeptide may exist in single or multiple copies within the genome of an individual. Such duplicate genes may be identical or may have certain modifications, including nucleotide substitutions, additions or deletions, which all still code for polypeptides having substantially the same activity. The term “DNA sequence encoding a SSP-1 polypeptide” may thus refer to one or more genes within a particular individual. Moreover, certain differences in nucleotide sequences may exist between individual organisms, which are called alleles. Such allelic differences may or may not result in differences in amino acid sequence of the encoded polypeptide yet still encode a protein with the same biological activity.

As used herein, the term “equivalent” includes nucleotide sequences encoding functionally equivalent SSP-1 polypeptides or functionally equivalent peptides having an activity of a SSP-1 protein such as described herein. Equivalent nucleotide sequences will include sequences that differ by one or more nucleotide substitution, addition or deletion, such as allelic variants; and will, therefore, include sequences that differ from the nucleotide sequence of the SSP-1 gene shown in SEQ ID NOS: 1 or 3 due to the degeneracy of the genetic code.

As used herein, the term “gene” or “recombinant gene” refers to a nucleic acid molecule comprising an open reading frame encoding one of the SSP-1 polypeptides of the present invention. A “recombinant gene” refers to nucleic acid molecule encoding a SSP-1 polypeptide and comprising SSP-1 protein-encoding exon sequences, though it may optionally include intron sequences which are either derived from a chromosomal SSP-1 gene or from an unrelated chromosomal gene. Exemplary recombinant genes encoding the subject SSP-1 polypeptides are represented in the appended Sequence Listing. The term “intron” refers to a DNA sequence present in a given gene which is not included in the mature mRNA and is generally found between exons.

A disease, disorder or condition “associated with” or “characterized by” an aberrant SSP-1 activity refers to a disease, disorder or condition in a subject which is caused by or contributed to by an aberrant SSP-1 activity.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the SSP-1 sequences of the present invention.

The term “interact” as used herein is meant to include detectable interactions between molecules, such as can be detected using, for example, a yeast two hybrid assay. The term interact is also meant to include “binding” interactions between molecules. Interactions may be protein-protein or protein-nucleic acid or protein-small molecule or nucleic acid-small molecule in nature.

The term “isolated” as used herein with respect to nucleic acids, such as DNA or RNA, refers to molecules separated from other DNAs or RNAs, respectively, that are present in the natural source of the macromolecule. For example, an isolated nucleic acid encoding one of the subject SSP-1 polypeptides preferably includes no more than 10 kilobases (kb) of nucleic acid sequence which naturally immediately flanks the SSP-1 gene in genomic DNA, more preferably no more than 5 kb of such naturally occurring flanking sequences, and most preferably less than 1.5 kb of such naturally occurring flanking sequence. Moreover, an “isolated nucleic acid” is meant to include nucleic acid fragments which are not naturally occurring as fragments and would not be found in the natural state. The term isolated as used herein also refers to a nucleic acid or peptide that is substantially free of cellular material, viral material, or culture medium when produced by recombinant DNA techniques, or chemical precursors or other chemicals when chemically synthesized. The term “isolated” is also used herein to refer to polypeptides which are isolated from other cellular proteins and is meant to encompass both purified and recombinant polypeptides.

“Modulation” as used herein is meant to encompass up-regulation (e.g., stimulation or activation) or down-regulation (e.g., inhibition or suppression) of bioactivity, such as gene expression in a cell expression a bioactivity of SSP-1. Modulating agents of the present invention can be nucleic acids, polypeptides, antibodies, or compounds.

“Non-human animals” of the invention include mammalians such as rodents, non-human primates, sheep, dog, cow, chickens, amphibians, reptiles, etc. Preferred non-human animals are selected from the rodent family including rat and mouse, most preferably mouse. The term “chimeric animal” is used herein to refer to animals in which the recombinant gene is found, or in which the recombinant gene is expressed in some but not all cells of the animal. The term “tissue-specific chimeric animal” indicates that one of the recombinant SSP- 1 genes is present and/or expressed or disrupted in some tissues but not others.

As used herein, the term “nucleic acid” refers to polynucleotides such as deoxyribonucleic acid (DNA), and, where appropriate, ribonucleic acid (RNA) and include antisense compounds and ribozymes. The term should also be understood to include, as equivalents, degenerate variants, analogs of either RNA or DNA made from nucleotide analogs, and, as applicable to the embodiment being described, single (sense or antisense) and double-stranded polynucleotides.

As used herein, the term “promoter” means a DNA sequence that regulates expression of a selected DNA sequence operably linked to the promoter, and which effects expression of the selected DNA sequence in cells. As used herein, the term “operably linked” is intended to mean that the nucleotide sequence is linked to a regulatory sequence in a manner which allows expression of the nucleotide sequence. The term encompasses “tissue specific” promoters, i.e. promoters, which effect expression of the selected DNA sequence only in specific cells (e.g., cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The terms “protein”, “polypeptide” and “peptide” are used interchangably herein when referring to a gene product.

The term “purified” refers to a peptide or DNA or RNA sequence that is present in the substantial absence of other biological macromolecules, such as other proteins. The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either natural materials in their native state or natural materials that have been separated into components (e.g., in an acrylamide gel) but not obtained either as pure (e.g., lacking contaminating proteins, or chromatography reagents such as denaturing agents and polymers, e.g., acrylamide or agarose) substances or solutions. In preferred embodiments, purified SSP-1 preparations will lack any contaminating proteins from the same animal from which SSP-1 is normally produced, as can be accomplished by recombinant expression of, for example, a human SSP-1 protein in a non-human cell.

The term “recombinant protein” refers to a polypeptide of the present invention which is produced by recombinant DNA techniques, wherein generally, DNA encoding a SSP-1 polypeptide is inserted into a suitable expression vector which is in turn used to transform a host cell to produce the heterologous protein. Moreover, the phrase “derived from”, with respect to a recombinant SSP-1 gene, is meant to include within the meaning of “recombinant protein” those proteins having an amino acid sequence of a native SSP-1 protein, or an amino acid sequence similar thereto which is generated by mutations including substitutions and deletions (including truncation) of a naturally occurring form of the protein.

As used herein, the term “specifically hybridizes” or “specifically detects” refers to the ability of a nucleic acid molecule of the invention to hybridize to at least approximately 6, 12, 20, 30, 50, 100, 150, 200, 300, 350, 400 or 425 consecutive nucleotides of a vertebrate, preferably mammalian, SSP-1 gene, such as the SSP-1 sequence designated in SEQ ID NOS: 1 or 3, or a sequence complementary thereto, or naturally occurring mutants thereof, such that it shows 10 times more hybridization, preferably more than 100 times more hybridization, and even more preferably more than 100 times more hybridization than it does to a cellular nucleic acid (e.g., mRNA or genomic DNA) encoding a protein other than a vertebrate, preferably mammalian, SSP-1 protein as defined herein.

The term “SSP-1 receptor” refers to a protein or protein complex, to which an SSP-1 protein, e.g., human SSP-1, can bind. A receptor can be a cell surface receptor, e.g., a nuclear hormone receptor. SSP-1 receptors can be isolated by methods known in the art and further described herein. Interaction of an SSP-1 protein with an SSP-1 receptor can result in transduction of a signal from the cell surface to the nucleus. The signal transduced can be, an increase in intracellular calcium, an increase in phosphatidylinositol or other molecule, and can result, e.g., in phosphorylation of specific proteins, a modulation of gene transcription and any of the other biological activities set forth herein.

The term “SSP-1 therapeutic” refers to various forms of SSP-1 polypeptides, as well as peptidomimetics, which can modulate at least one activity of a SSP-1 protein, e.g., binding to an SSP-1 receptor or inducing transduction of an intracellular signal, by mimicking or potentiating (agonizing) or inhibiting (antagonizing) the effects of a naturally-occurring SSP-1 protein. A SSP-1 therapeutic which mimics or potentiates the activity of a wild-type SSP-1 protein is a “SSP-1 agonist”. Conversely, a SSP-1 therapeutic which inhibits the activity of a wild-type SSP-1 protein is a “SSP-1 antagonist”.

The terms “SSP-1 polypeptide” and “SSP-1 protein” are intended to encompass polypeptides comprising the amino acid sequence SEQ ID No. 2, fragments thereof, and homologs thereto and include agonist and antagonist polypeptides.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the recombinant SSP-1 genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of SSP-1 proteins.

As used herein, the term “transfection” means the introduction of a nucleic acid, e.g., an expression vector, into a recipient cell by nucleic acid-mediated gene transfer whether or not any coding sequences are ultimately expressed. Cells do not naturally take up DNA. Methods of transfection are known to the ordinarily skilled artisan, and include CaPO4, electroporation and DEAE Dextran. (J. Sambrook, E. Fritsch, T. Maniatis, Molecular Cloning: A Laboratory Manual, Cold Spring Laboratory Press, 1989.) As used herein, the term “transgene” means a nucleic acid sequence encoding, e.g., one of the SSP-1 polypeptides, or an antisense transcript thereto, which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, (e.g., as intron), that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the SSP-1 proteins, e.g., either agonistic or antagonistic forms. However, transgenic animals in which the recombinant SSP-1 gene is silent are also contemplated, as for example, the FLP or CRE recombinase dependent constructs described below. Moreover, “transgenic animal” also includes those recombinant animals in which gene disruption of one or more SSP-1 genes is caused by human intervention, including both recombination and antisense techniques.

The term “treating” as used herein is intended to encompass curing as well as ameliorating at least one symptom of the condition or disease.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as plasmids are the most commonly used form of vectors. However, the invention is intended to include such other forms of expression vectors which serve equivalent functions and which become known in the art subsequently hereto.

Nucleic Acids of the Present Invention

As described below, one aspect of the invention pertains to isolated nucleic acids comprising nucleotide sequences encoding SSP-1 polypeptides, degenerate variants and/or equivalents of such nucleic acids. The term equivalent is understood to include nucleotide sequences encoding functionally equivalent SSP-1 polypeptides or functionally equivalent peptides having an activity of a SSP-1 protein such as described herein. Equivalent nucleotide sequences will include sequences that differ by one or more nucleotide substitution, addition or deletion, such as allelic variants; and will, therefore, include sequences that differ from the nucleotide sequence of the SSP-1 gene shown in SEQ ID NOS: 1 or 3 due to the degeneracy of the genetic code.

Preferred nucleic acids are vertebrate SSP-1 nucleic acids. Particularly preferred vertebrate SSP-1 nucleic acids are mammalian. Regardless of species, particularly preferred SSP-1 nucleic acids encode polypeptides that are at least 70%, 80%, 90%, or 95% similar to an amino acid sequence of a vertebrate SSP-1 protein. In one embodiment, the nucleic acid is a cDNA encoding a polypeptide having at least one bio-activity of the subject SSP-1 polypeptide. Preferably, the nucleic acid includes all or a portion of the nucleotide sequence corresponding to the nucleic acid of SEQ ID No 1 or 3.

Still other preferred nucleic acids of the present invention encode a SSP-1 polypeptide which is comprised of at least 2, 5, 10, 25, 50, 100, 150 or 200 amino acid residues. For example, preferred nucleic acid molecules for use as probes/primer or antisense molecules (i.e. noncoding nucleic acid molecules) can comprise at least about 6, 12, 20, 30, 50, 60, 70, 80, 90 or 100 base pairs in length, whereas coding nucleic acid molecules can comprise about 50, 60, 70, 80, 90, or 100 base pairs.

Another aspect of the invention provides a nucleic acid which hybridizes under stringent conditions to a nucleic acid represented by SEQ ID NOS: I or 3. Appropriate stringency conditions which promote DNA hybridization, for example, 6.0× sodium chloride/sodium citrate (SSC) at about 45° C., followed by a wash of 2.0× SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0× SSC at 50° C. to a high stringency of about 0.2× SSC at 50° C. In addition, the temperature in the wash step can be increased from low stringency conditions at room temperature, about 22° C., to high stringency conditions at about 65° C. Both temperature and salt may be varied, or temperature of salt concentration may be held constant while the other variable is changed. In a preferred embodiment, a SSP-1 nucleic acid of the present invention will bind to one of SEQ ID NOS 1 or 3 under moderately stringent conditions, for example at about 2.0× SSC and about 40° C. In a particularly preferred embodiment, a SSP-1 nucleic acid of the present invention will bind to one of SEQ ID NOS: 1 or 3 under high stringency conditions.

Preferred nucleic acids have a sequence at least 70%, and more preferably 75% homologous and more preferably 80% and even more preferably at least 85% homologous with an amino acid sequence of a SSP-1 gene, e.g., such as a sequence shown in one of SEQ ID NOS: 1 or 3. Nucleic acids at least 90%, more preferably 95%, and most preferably at least about 98-99% homologous with a nucleic sequence represented in one of SEQ ID NOS: 1 or 3 are of course also within the scope of the invention. In preferred embodiments, the nucleic acid is mammalian and in particularly preferred embodiments, includes all or a portion of the nucleotide sequence corresponding to the coding region of one of SEQ ID NOS: 1 or 3.

Nucleic acids having a sequence that differs from the nucleotide sequences shown in one of SEQ ID NOS: 1 or 3 due to degeneracy in the genetic code are also within the scope of the invention. Such nucleic acids encode functionally equivalent peptides (i.e., a peptide having a biological activity of a SSP-1 polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of a SSP-1 polypeptide. However, it is expected that DNA sequence polymorphisms that do lead to changes in the amino acid sequences of the subject SSP-1 polypeptides will exist among mammals. One skilled in the art will appreciate that these variations in one or more nucleotides (e.g., up to about 3-5% of the nucleotides) of the nucleic acids encoding polypeptides having an activity of a SSP-1 polypeptide may exist among individuals of a given species due to natural allelic variation.

The polynucleotide sequence of the present invention may encode for a mature form of the SSP-1. The polynucleotide sequence may also encode for a leader sequence. For example, the desired DNA sequence may be fused in the same reading frame to a DNA sequence which aids in expression and secretion of the polypeptide from the host cell, for example, a leader sequence which functions as a secretory sequence for controlling transport of the polypeptide from the cell. The protein having a leader sequence is a preprotein and may have the leader sequence cleaved by the host cell to form the mature form of the protein. The polynucleotide of the present invention may also be fused in frame to a marker sequence which allows for purification of the polypeptide of the present invention.

In a preferred embodiment, the marker sequence is a hexahistidine tag supplied by a PQE-9 vector to provide for purification of the fusion protein in the case of a bacterial host or an HA tag when a mammalian host, e.g. COS-7 cells, are used.

As indicated by the examples set out below, SSP-1 protein-encoding nucleic acids can be obtained from mRNA present in any of a number of eukaryotic cells. It should also be possible to obtain nucleic acids encoding SSP-1 polypeptides of the present invention from genomic DNA from both adults and embryos. For example, a gene encoding a SSP-1 protein can be cloned from either a cDNA or a genomic library in accordance with protocols described herein, as well as those generally known to persons skilled in the art. Examples of tissues and/or libraries suitable for isolation of the subject nucleic acids include thymus, lymph nodes and inflammatory tissue. cDNA encoding a SSP-1 protein can be obtained by isolating total mRNA from a cell, e.g., a vertebrate cell, a mammalian cell, or a human cell, including embryonic cells. Double stranded cDNAs can then be prepared from the total mRNA, and subsequently inserted into a suitable plasmid or bacteriophage vector using any one of a number of known techniques. The gene encoding a SSP-1 protein can also be cloned using established polymerase chain reaction techniques in accordance with the nucleotide sequence information provided by the invention. The nucleic acid of the invention can be DNA or RNA or analogs thereof. A preferred nucleic acid is a cDNA represented by a sequence selected from the group consisting of SEQ ID NOS: 1 or 3.

Preferred nucleic acids encode a vertebrate SSP-1 polypeptide comprising an amino acid sequence at least 60% homologous, more preferably 70% homologous and most preferably 80% homologous with an amino acid sequence contained in SEQ ID No: 2. Nucleic acids which encode polypeptides at least about 90%, more preferably at least about 95%, and most preferably at least about 98-99% homology with an amino acid sequence represented in SEQ ID No: 2 are also within the scope of the invention. In one embodiment, the nucleic acid is a cDNA encoding a peptide having at least one activity of the subject vertebrate SSP-1 polypeptide. Preferably, the nucleic acid includes all or a portion of the nucleotide sequence corresponding to the coding region of SEQ ID NOS: I and 3.

Preferred nucleic acids encode a bioactive fragment of a vertebrate SSP-1 polypeptide comprising an amino acid sequence at least 60% homologous, more preferably 70% homologous and most preferably 80% homologous with an amino acid sequence selected from the group consisting of SEQ ID No: 2. Nucleic acids which encode polypeptides at least about 90%, more preferably at least about 95%, and most preferably at least about 98-99% homology, or identical, with an amino acid sequence represented in one of SEQ ID No: 2 are also within the scope of the invention.

Vectors

This invention also provides expression vectors containing a nucleic acid encoding a SSP-1 polypeptide, operably linked to at least one transcriptional regulatory sequence. Regulatory sequences are art-recognized and are selected to direct expression of the subject SSP-1 proteins. Transcriptional regulatory sequences are described in Goeddel; Gene Expression Technology: Methods in Enzymology 185, Academic Press, San Diego, Calif. (1990). In one embodiment, the expression vector includes a recombinant gene encoding a peptide having an agonistic activity of a subject SSP-1 polypeptide, or alternatively, encoding a peptide which is an antagonistic form of the SSP-1 protein. Such expression vectors can be used to transfect cells and thereby produce polypeptides, including fusion proteins, encoded by nucleic acids as described herein. Moreover, the gene constructs of the present invention can also be used as a part of a gene therapy protocol to deliver nucleic acids encoding either an agonistic or antagonistic form of one of the subject SSP-1 proteins. Thus, another aspect of the invention features expression vectors for in vivo or in vitro transfection and expression of a SSP-1 polypeptide in particular cell types so as to reconstitute the function of, or alternatively, abrogate the function of SSP-1 induced signaling in a tissue. This could be desirable, for example, when the naturally-occurring form of the protein is misexpressed; or to deliver a form of the protein which alters differentiation of tissue. Expression vectors may also be employed to inhibit neoplastic transformation.

In addition to viral transfer methods, such as those illustrated above, nonviral methods can also be employed to cause expression of a subject SSP-1 polypeptide in the tissue of an animal. Most nonviral methods of gene transfer rely on normal mechanisms used by mammalian cells for the uptake and intracellular transport of macromolecules. In preferred embodiments, non-viral targeting means of the present invention rely on endocytic pathways for the uptake of the subject SSP-1 polypeptide gene by the targeted cell. Exemplary targeting means of this type include liposomal derived systems, poly-lysine conjugates, and artificial viral envelopes.

Probes and Primers

The nucleotide sequences determined from the cloning of SSP-1 genes from mammalian organisms will further allow for the generation of probes and primers designed for use in identifying and/or cloning SSP-1 homologs in other cell types, e.g., from other tissues, as well as SSP-1 homologs from other mammalian organisms. For instance, the present invention also provides a probe/primer comprising a substantially purified oligonucleotide, which oligonucleotide comprises a region of nucleotide sequence that hybridizes under stringent conditions to at least approximately 12, preferably 25, more preferably 40, 50 or 75 consecutive nucleotides of sense or anti-sense sequence selected from the group consisting of SEQ ID No: 1 or 3 or naturally occurring mutants thereof. For instance, primers based on the nucleic acid represented in SEQ ID NOs: 1 or 3 can be used in PCR reactions to clone SSP-1 homologs.

Likewise, probes based on the subject SSP-1 sequences can be used to detect transcripts or genomic sequences encoding the same or homologous proteins. In preferred embodiments, the probe further comprises a label group attached thereto and able to be detected, e.g., the label group is selected from amongst radioisotopes, fluorescent compounds, enzymes, and enzyme co-factors.

Antisense. Ribozyme and Triplex techniques

Another aspect of the invention relates to the use of the isolated nucleic acid in “antisense” therapy. As used herein, “antisense” therapy refers to administration or in situ generation of oligonucleotide molecules or their derivatives which specifically hybridize (e.g., bind) under cellular conditions, with the cellular mRNA and/or genomic DNA encoding one or more of the subject SSP-1 proteins so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a SSP-1 protein. Alternatively, the antisense construct is an oligonucleotide probe which is generated ex vivo and which, when introduced into the cell causes inhibition of expression by hybridizing with the mRNA and/or genornic sequences of a SSP-1 gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the SSP-1 nucleotide sequence of interest, are preferred.

Antisense approaches involve the design of oligonucleotides (either DNA or RNA) that are complementary to SSP-1 mRNA. The antisense oligonucleotides will bind to the SSP-1 mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, noncoding regions of a SSP-1 gene could be used in an antisense approach to inhibit translation of endogenous SSP-1 mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of SSP-1 mRNA, antisense nucleic acids should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense oligonucleotide to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. the oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine,

5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The. oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate olgonucleotides can be prepared by use of controlled pore glass polymer supports (Sarin et al., 1988, Proc. Natl. Acad. Sci. U.S.A. 85:7448-7451), etc.

While antisense nucleotides complementary to the SSP-1 coding region sequence can be used, those complementary to the transcribed untranslated region and to the region comprising the initiating methionine are most preferred.

The antisense molecules can be delivered to cells which express SSP-1 in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it is often difficult to achieve intracellular concentrations of the antisense sufficient to suppress translation on endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous SSP-1 transcripts and thereby prevent translation of the SSP-1 MRNA, For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct which can be introduced directly into the tissue site; e.g., the choroid plexus or hypothalamus. Alternatively, viral vectors can be used which selectively infect the desired tissue; (e.g., for brain, herpesvirus vectors may be used), in which case administration may be accomplished by another route (e.g., systematically).

Ribozyme molecules designed to catalytically cleave SSP-1 mRNA transcripts can also be used to prevent translation of SSP-1 mRNA and expression of SSP-1 (See, e.g., PCT International Publication WO90/11364, published Oct. 4, 1990; Sarver et al., 1990, Science 247:1222-1225 and U.S. Pat. No. 5,093,246). While ribozymes that cleave mRNA at site specific recognition sequences can be used to destroy SSP-1 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. There are a number of potential hammerhead ribozyme cleavage sites within the nucleotide sequence of human SSP-1 cDNA (FIG. 1). Preferably the ribozyme is engineered so that the cleavage recognition site is located near the 5′ end of the SSP-1 mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a SSP-1 gene.

As in the antisense approach, the ribozymes can be composed of modified oligonucleotides (e.g., for improved stability, targeting, etc.) and should be delivered to cells which express the SSP-1 gene in vivo. A preferred method of delivery involves using a DNA construct “encoding” the robozyme under the control of a strong constitutive pol III or pol II promoter, so that transfected cells will produce sufficient quantities of the ribozyme to destroy endogenous SSP-1 messages and inhibit translation. Because ribozymes unlike antisense molecules, are catalytic, a lower intracellular concentration is required for efficiency.

Endogenous SSP-1 gene expression can also be reduced by inactivating or “knocking out” the SSP-1 gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional SSP-1 (or a completely unrelated DNA sequence) flanked by DNA homologous to the endogenous SSP-1 gene (either the coding regions or regulatory regions of the SSP-1 gene) can be used, with or without a selectable marker and/or a negative selectable marker, to transfect cells that express SSP-1 in vivo. Insertion of the DNA construct, via targeted homologous recombination, results in inactivation of the SSP-1 gene. Such approaches are particularly suited in the agricultural field where modifications to ES (embryonic stem) cells can be used to generate animal offspring with an inactive SSP-1 (e.g., see Thomas & Capecchi 1987 and Thompson 1989, supra). However this approach can be adapted for use in humans provided the recombinant DNA constructs are directly administered or targeted to the required site in vivo using appropriate viral vectors, e.g., herpes virus vectors for delivery to brain tissue; e.g., the hypothalamus and/or choroid plexus.

Alternatively, endogenous SSP-1 gene expression can be reduced by targeting deoxyribonucleotide sequences complementary to the regulatory region of the SSP-1 gene (i.e., the SSP-1 promoter and/or enhancers) to form triple helical structures that prevent transcription of the SSP- I gene in target cells in the body. (See generally, Helene, C. 1991, Anticancer Drug Des., 6(6):569-84; Helene, C., et al., 1992, Ann, N.Y. Accad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ )-methyl rather than phosphodiesterase linkages within the oligodeoxyribonucleotide backbone.

Polypeptides of the Present Invention

The present invention also makes available isolated SSP-1 polypeptides which are isolated from, or otherwise substantially free of other cellular proteins, especially other signal transduction factors and/or transcription factors which may normally be associated with the SSP-1 polypeptide. The term “substantially free of other cellular proteins” (also referred to herein as “contaminating proteins”) or “substantially pure or purified preparations” are defined as encompassing preparations of SSP-1 polypeptides having less than about 20% (by dry weight) contaminating protein, and preferably having less than about 5% contaminating protein. Functional forms of the subject polypeptides can be prepared, for the first time, as purified preparations by using a cloned gene as described herein.

Full length proteins or fragments corresponding to one or more particular motifs and/or domains or to arbitrary sizes, for example, at least 5, 10, 25, 50, 75 and 100, amino acids in length are within the scope of the present invention.

For example, isolated SSP-1 polypeptides can be encoded by all or a portion of a nucleic acid sequence shown in any of SEQ ID NOS. 1 or 3. Isolated peptidyl portions of SSP-1 proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a SSP-1 polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) SSP-1 protein.

Another aspect of the present invention concerns recombinant forms of the SSP-1 proteins. Recombinant polypeptides preferred by the present invention, in addition to native SSP-1 proteins (e.g., as set forth in SEQ ID NO: 3), are encoded by a nucleic acid, which is at least 85% homologous and more preferably 90% homologous and most preferably 95% homologous with an amino acid sequence represented by SEQ ID No: 2 or encoded by SEQ ID NOS. 1 or 3. Polypeptides which are encoded by a nucleic acid that is at least about 98-99% homologous with the sequence of SEQ ID NOS: 1 or 3 or which are 98-99% homologous with the amino acid sequence set forth in SEQ ID NO: 2 are also within the scope of the invention. In a preferred embodiment, a SSP-1 protein of the present invention is a mammalian SSP-1 protein. In a particularly preferred embodiment a SSP-1 protein is set forth as SEQ ID No: 2. In particularly preferred embodiment, a SSP-1 protein has a SSP-1 bioactivity. It will be understood that certain post-translational modifications, e.g., phosphorylation and the like, can increase the apparent molecular weight of the SSP-1 protein relative to the unmodified polypeptide chain.

The present invention further pertains to recombinant forms of one of the subject SSP-1 polypeptides. Such recombinant SSP-1 polypeptides preferably are capable of functioning in one of either role of an agonist or antagonist of at least one biological activity of a wild-type (“authentic”) SSP-1 protein of the appended sequence listing. The term “evolutionarily related to”, with respect to amino acid sequences of SSP-1 proteins, refers to both polypeptides having amino acid sequences which have arisen naturally, and also to mutational variants of human SSP-1 polypeptides which are derived, for example, by combinatorial mutagenesis.

In general, polypeptides referred to herein as having an activity (e.g., are “bioactive”) of a SSP-1 protein are defined as polypeptides which include an amino acid sequence encoded by all or a portion of the nucleic acid sequences shown in one of SEQ ID NOS: 1 or 3 and which mimic or antagonize all or a portion of the biological/biochemical activities of a naturally occurring SSP-1 protein. Examples of such biological activity include the ability to regulate cell growth, differentiation and/or survival. Other biological activities of the subject SSP-1 proteins are described herein or will be reasonably apparent to those skilled in the art. According to the present invention, a polypeptide has biological activity if it is a specific agonist or antagonist of a naturally-occurring form of a SSP-1 protein.

Assays for determining whether a compound, e.g, a protein, such as an SSP-1 protein or variant thereof, is capable of modulating cell growth, differentiation, and/or survival are well known in the art. For example, various amounts of the subject compound can be added to different types of cell lines or primary cells, and cell growth monitored by, e.g., counting the cells under a microscope or by using a Coulter Counter. Alternatively, thymidine incorporation assays can be performed.

To determine whether an SSP-1 protein or variant thereof is capable of modulating cell differentiation, various amounts of the SSP-1 protein or variant thereof can be added to cells which are capable of differentiation under appropriate conditions. Such cells include, e.g., 10T1/2 cells, which are capable of differentiating into different types of cells depending on the agent added to the culture. Yet other cells lines which can be used include 3T3-L1 cells which are capable of differentiating into fat cells.

To determine whether an SSP-1 protein or variant thereof is capable of modulating cell survival, e.g., apoptosis, various amounts of the SSP-1 protein or variant thereof can be added to a cell culture and the degree of cell death, e.g., apoptosis measured by methods known in the art, such as methods involving electrophoresis of genomic DNA from cells. Alternatively, the degree of apoptosis can be determined morphologically by using a microscope with, e.g., a 300 fold magnification.

The present invention further pertains to methods of producing the subject SSP-1 polypeptides. For example, a host cell transfected with a nucleic acid vector directing expression of a nucleotide sequence encoding the subject polypeptides can be cultured under appropriate conditions to allow expression of the peptide to occur. The SSP-1 protein can then from the supernatant of the cell culture be harvested, lysed and the protein isolated. A cell culture includes host cells, media and other byproducts. Suitable media for cell culture are well known in the art. The recombinant SSP-1 polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant SSP-1 polypeptide is a fusion protein containing a domain which facilitates its purification, such as GST fusion protein.

Moreover, it will be generally appreciated that, under certain circumstances, it may be advantageous to provide homologs of one of the subject SSP-1 polypeptides which function in a limited capacity as one of either a SSP-1 agonist (mimetic) or a SSP-1 antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of SSP-1 proteins.

Homologs of each of the subject SSP-1 proteins can be generated by mutagenesis, such as by discrete point mutation(s), or by truncation. For instance, mutation can give rise to homologs which retain substantially the same, or merely a subset, of the biological activity of the SSP-1 polypeptide from which it was derived. Alternatively, antagonistic forms of the protein can be generated which are able to inhibit the function of the naturally occurring form of the protein, such as by competitively binding to an SSP-1 receptor.

The recombinant SSP-1 polypeptides of the present invention also include homologs of the wildtype SSP-1 proteins, such as versions of those protein which are resistant to proteolytic cleavage, as for example, due to mutations which alter ubiquitination or other enzymatic targeting associated with the protein.

SSP-1 polypeptides may also be chemically modified to create SSP-1 derivatives by forming covalent or aggregate conjugates with other chemical moieties, such as glycosyl groups, lipids, phosphate, acetyl groups and the like. Covalent derivatives of SSP-1 proteins can be prepared by linking the chemical moieties to functional groups on amino acid sidechains of the protein or at the N-terminus or at the C-terminus of the polypeptide.

Modification of the structure of the subject SSP-1 polypeptides can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the SSP-1 polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2^{nd} ed., Ed. by L. Stryer, W H Freeman and Co.: 1981). Whether a change in the amino acid sequence of a peptide results in a functional SSP-1 homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

This invention further contemplates a method for generating sets of combinatorial mutants of the subject SSP-1 proteins as well as truncation mutants, and is especially useful for identifying potential variant sequences (e.g., homologs). The purpose of screening such combinatorial libraries is to generate, for example, novel SSP-1 homologs which can act as either agonists or antagonist, or alternatively, possess novel activities all together. Thus, combinatorially-derived homologs can be generated to have an increased potency relative to a naturally occurring form of the protein.

In one embodiment, the variegated library of SSP-1 variants is generated by combinatorial mutagenesis at the nucleic acid level, and is encoded by a variegated gene library. For instance, a mixture of synthetic oligonucleotides can be enzymatically ligated into gene sequences such that the degenerate set of potential SSP-1 sequences are expressible as individual polypeptides, or alternatively, as a set of larger fusion proteins (e.g., for phage display) containing the set of SSP-1 sequences therein.

There are many ways by which such libraries of potential SSP-1 homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential SSP-1 sequences. The synthesis of degenerate oligonucleotides is well known in the art (see for example, Narang, SA (1983) Tetrahedron 39:3; Itakura et al. (1981) Recombinant DNA, Proc 3^{rd} Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87:6378-6382; as well as U.S. Pat. Nos. 5,223,409, 5,198,346, and 5,096,815).

Likewise, a library of coding sequence fragments can be provided for a SSP-1 clone in order to generate a variegated population of SSP-1 fragments for screening and subsequent selection of bioactive fragments. A variety of techniques are known in the art for generating such libraries, including chemical synthesis. In one embodiment, a library of coding sequence fragments can be generated by (i) treating a double stranded PCR fragment of a SSP-1 coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of SSP-1 homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate SSP-1 sequences created by combinatorial mutagenesis techniques.

Combinatorial mutagenesis has a potential to generate very large libraries of mutant proteins, e.g., in the order of 10²⁶ molecules. Combinatorial libraries of this size may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the SSP-1 proteins to generate mimetics, e.g., peptide or non-pepide agents, such as small molecules, which are able to disrupt binding of a SSP-1 polypeptide of the present invention with a nucleotide, such as proteins, e.g. receptors. Thus, such mutagenic techniques as described above are also useful to map the determinants of the SSP-1 proteins which participate in proteinprotein interactions involved in, for example, binding of the subject SSP-1 polypeptide to its receptor. To illustrate, the critical residues of a subject SSP-1 polypeptide which are involved in molecular recognition of its receptor can be determined and used to generate SSP-1 derived peptidomimetics or small molecules which competitively inhibit binding of the authentic SSP-1 protein with that moiety. By employing, for example, scanning mutagenesis to map the amino acid residues of the subject SSP-1 proteins which are involved in binding other proteins, peptidomimetic compounds can be generated which mimic those residues of the SSP-1 protein which facilitate the interaction. Such mimetics may then be used to interfere with the normal function of a SSP-1 protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), ketomethylene pseudopeptides (Ewenson et al. (1986) J Med Chem 29:295; and Ewenson et al. in Peptides: Structure and Function (Proceedings of the 9^{th} American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

Cells expressing recombinant SSP-1 polypeptides

This invention also pertains to host cells transfected to express a recombinant form of the subject SSP-1 polypeptides. The host cell may be any prokaryotic or eukaryotic cell. Thus, a nucleotide sequence derived from the cloning of mammalian SSP-1 proteins, encoding all or a selected portion of the full-length protein, can be used to produce a recombinant form of a SSP-1 polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures used in producing other well-known proteins, e.g., MAP kinase, p53, WT1, PTP phosphotases, SRC, and the like. Similar procedures, or modifications thereof, can be employed to prepare recombinant SSP-1 polypeptides by microbial means or tissue-culture technology in accord with the subject invention.

The recombinant SSP-1 genes can be produced by ligating a nucleic acid encoding a SSP-1 protein, or a portion thereof, into a vector suitable for expression in either prokaryotic cells, eukaryotic cells, or both. Expression vectors for production of recombinant forms of the subject SSP-1 polypeptides include plasmids and other vectors. For instance, suitable vectors for the expression of a SSP-1 polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used. In an illustrative embodiment, a SSP-1 polypeptide is produced recombinantly utilizing an expression vector generated by sub-cloning the coding sequence of one of the SSP-1 genes represented in SEQ ID NOs: 1 or 3.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press:1989) Chapters 16 and 17.

In some instances, it may be desirable to express the recombinant SSP-1 polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III) When it is desirable to express only a portion of a SSP-1 protein, such as a form lacking a portion of the N-terminus, i.e. a truncation mutant which lacks the signal peptide, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing SSP-1 derived polypeptides in a host which produces MAP (e.g., E. coli or CM89 or S. cerevisiae), or in vitro by use of purified MAP (e.g., procedure of Miller et al., supra).

In other embodiments transgenic animals, described in more detail below could be used to produce recombinant proteins.

Fusion proteins and Immunogens

In another embodiment, the coding sequences for the polypeptide can be incorporated as a part of a fusion gene including a nucleotide sequence encoding a different polypeptide. This type of expression system can be useful under conditions where it is desirable to produce an immunogenic fragment of a SSP-1 protein. For example, the VP6 capsid protein of rotavirus can be used as an immunologic carrier protein for portions of the SSP-1 polypeptide, either in the monomeric form or in the form of a viral particle. The nucleic acid sequences corresponding to the portion of a subject SSP-1 protein to which antibodies are to be raised can be incorporated into a fusion gene construct which includes coding sequences for a late vaccinia virus structural protein to produce a set of recombinant viruses expressing fusion proteins comprising SSP-1 epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of a SSP-1 protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of a SSP-1 polypeptide is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of SSP-1 proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the SSP-1 polypeptides of the present invention. For example, SSP-1 polypeptides can be generated as glutathione-S-transferase (GST-fusion) proteins. Such GST-fusion proteins can enable easy purification of the SSP-1 polypeptide, as for example by the use of glutathionederivatized matrices (see, for example, Current Protocols in Molecular Biology, eds. Ausubel et al. (N.Y.: John Wiley & Sons, 1991)).

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²+ metal resin. The purification leader sequence can then be subsequently removed by treatment with enterokinase to provide the purified protein (e.g., see Hochuli et al. (1987) J. Chromatography 411:177; and Janknecht et al. PNAS 88:8972).

Techniques for making fusion genes are known to those skilled in the art. Essentially, the joining of various DNA fragments coding for different polypeptide sequences is performed in accordance with conventional techniques, employing bluntended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al. John Wiley & Sons: 1992).

Antibodies

Another aspect of the invention pertains to an antibody specifically reactive with a mammalian SSP-1 protein. For example, by using immunogens derived from a SSP-1 protein, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a mammalian SSP-1 polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of a SSP-1 protein can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of a SSP-1 protein of a mammal, e.g., antigenic determinants of a protein set forth in SEQ ID No: 2 or closely related homologs (e.g., at least 90% homologous, and more preferably at least 94% homologous).

Following immunization of an animal with an antigenic preparation of a SSP-1 polypeptide, anti- SSP-1 antisera can be obtained and, if desired, polyclonal anti-SSP-1 antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian SSP-1 polypeptide of the present invention and monoclonal antibodies isolated from a culture comprising such hybridoma cells. In one embodiment anti-human SSP-1 antibodies specifically react with the protein encoded by the DNA of ATCC deposit No.

The term antibody as used herein is intended to include fragments thereof which are also specifically reactive with one of the subject mammalian SSP-1 polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a SSP-1 protein conferred by at least one CDR region of the antibody. In preferred embodiments, the antibodies, the antibody further comprises a label attached thereto and able to be detected, (e.g., the label can be a radioisotope, fluorescent compound, enzyme or enzyme co-factor).

Anti-SSP-1 antibodies can be used, e.g., to monitor SSP-1 protein levels in an individual for determining, e.g., whether a subject has a disease or condition associated with an aberrant SSP-1 protein level, or allowing determination of the efficacy of a given treatment regimen for an individual afflicted with such a disorder. The level of SSP-1 polypeptides may be measured from cells in bodily fluid, such as in blood samples.

Another application of anti-SSP-1 antibodies of the present invention is in the immunological screening of cDNA libraries constructed in expression vectors such as λgt11, λgt18-23, λZAP, and λORF8. Messenger libraries of this type, having coding sequences inserted in the correct reading frame and orientation, can produce fusion proteins. For instance, λgt11 will produce fusion proteins whose amino termini consist of B-galactosidase amino acid sequences and whose carboxy termini consist of a foreign polypeptide. Antigenic epitopes of a SSP-1 protein, e.g., other orthologs of a particular SSP-1 protein or other paralogs from the same species, can then be detected with antibodies, as, for example, reacting nitrocellulose filters lifted from infected plates with anti-SSP-1 antibodies. Positive phage detected by this assay can then be isolated from the infected plate. Thus, the presence of SSP-1 homologs can be detected and cloned from other animals, as can alternate isoforms (including splicing variants) from humans.

Methods of Treating Disease

The invention provides methods for treating or preventing a disease caused by or contributed to by an aberrant SSP-1 activity in a subject, comprising administering to the subject an effective amount of a pharmaceutical composition comprising a compound which is capable of modulating a SSP-1 activity, such that the disease is treated or prevented in the subject. In one embodiment, the disease is a disease characterized by an abnormal cell proliferation, differentiation, and/or survival. For example, the disease can be a hyper- or hypoproliferative disease. The invention also provides methods for treating diseases characterized by an abnormal cell proliferation, differentiation, and/or survival in a subject, which are not characterized by an abnormal SSP-1 activity. In fact, since SSP-1 is likely to be capable of modulating the proliferative state of a cell (i.e., state of proliferation, differentiation, and or survival of a cell), SSP-1 can regulate disease wherein the abnormal proliferative state of a cell results from a defect other than an abnormal SSP-1 activity.

Hyperproliferative diseases that can be treated with SSP-1 therapeutics include neoplastic and hyperplastic diseases, such as various forms of cancers and leukemias, and fibroproliferative disorders. Other hyperproliferative diseases that can be treated or prevented with the subject SSP-1 therapeutics include malignant conditions, premalignant conditions, and benign conditions. The condition to be treated or prevented can be a solid tumor, such as a tumor arising in an epithelial tissue. Accordingly, treatment of such a cancer could comprise administration to the subject of a SSP-1 therapeutic decreasing the interaction of SSP-1 with an SSP-1 receptor. Other cancers that can be treated or prevented with a SSP-1 protein include sarcomas and carcinomas, e.g., lung cancer, cancer of the colon, prostate, breast, ovary, esophagus, lung cancer, melanoma, seminoma, and squamous adenocarcinoma. Additional solid tumors within the scope of the invention include those that can be found in a medical textbook.

The condition to be treated or prevented can also be a soluble tumor, such as leukemia, either chronic or acute, including chronic or acute myelogenous leukemia, chronic or acute lymphocytic leukemia, promyelocytic leukemia, monocytic leukemia, myelomonocytic leukemia, and erythroleukemia. Yet other proliferative disorders that can be treated with a SSP-1 therapeutic of the invention include heavy chain disease, multiple myeloma, lymphoma, e.g., Hodgkin's lymphoma and non-Hodgkin's lymphoma, and Waldenstroem's macroglobulemia.

Diseases or conditions characterized by a solid or soluble tumor can be treated by administrating a SSP-1 therapeutic either locally or systemically, such that aberrant cell proliferation is inhibited or decreased. Methods for administering the compounds of the invention are further described below.

The invention also provides methods for preventing the formation and/or development of tumors. For example, the development of a tumor can be preceded by the presence of a specific lesion, such as a pre-neoplastic lesion, e.g., hyperplasia, metaplasia, and dysplasia, which can be detected, e.g., by cytologic methods. Such lesions can be found, e.g., in epithelial tissue. Thus, the invention provides a method for inhibiting progression of such a lesion into a neoplastic lesion, comprising administering to the subject having a preneoplastic lesion an amount of a SSP-1 therapeutic sufficient to inhibit progression of the preneoplastic lesion into a neoplastic lesion.

The invention also provides for methods for treating or preventing diseases or conditions in which proliferation of cells is desired. For example, SSP-1 therapeutics can be used to stimulate tissue repair or wound healing, such as after surgery or to stimulate tissue healing from burns. Other diseases in which proliferation of cells is desired are hypoproliferative diseases, i.e, diseases characterized by an abnormally low proliferation of certain cells.

In yet another embodiment, the invention provides a method for treating or preventing diseases or conditions characterized by aberrant cell differentiation. Accordingly, the invention provides methods for stimulating cellular differentiation in conditions characterized by an inhibition of normal cell differentiation which may or may not be accompanied by excessive proliferation. Alternatively, SSP-1 therapeutics can be used to inhibit differentiation of specific cells.

In a preferred method, the aberrantly proliferating and/or differentiating cell is a cell present in the nervous system. A role for SSP-1 in the nervous system is suggested at least in part from the fact that human SSP-1 is expressed in human fetal brain. Accordingly, the invention provides methods for treating diseases or conditions associated with a central or peripheral nervous system. For example, the invention provides methods for treating lesions of the nervous system associated with an aberrant proliferation, differentiation or survival of any of the following cells: neurons, Schwann cells, glial cells, and other types of neural cells. Disorders of the nervous system include, but are not limited to: spinal cord injuries, brain injuries, lesions associated with surgery, ischemic lesions, malignant lesions, infectious lesions, degenerative lesions (Parkinson's disease, Alzheimer's disease, Huntington's chorea, amyotrophic lateral sclerosis), demyelating diseases (multiple sclerosis, human immunodeficiency associated myelophathy, transverse myelopathy, progressive multifocal leukoencephalopathy, pontine myelinolysis), motor neuron injuries, progressive spinal muscular atrophy, progressive bulbar palsy, primary lateral sclerosis, infantile and juvenile muscular atrophy, progressive bulbar paralysis of childhood (Fazio-Londe syndrome), poliomyelitis, and hereditary motorsensory neuropathy (Charcot-Marie-Tooth disease).

In another embodiment, the invention provides a method for enhancing the survival and/or stimulating proliferation and/or differentiation of cells and tissues in vitro In a preferred embodiment, SSP-1 therapeutics are used to promote tissue regeneration and/or repair (e.g., to treat nerve injury). For example, tissues from a subject can be obtained and grown in vitro in the presence of a SSP-1 therapeutic, such that the tissue cells are stimulated to proliferate and/or differentiate. The tissue can then be readministered to the subject.

Among the approaches which may be used to ameliorate disease symptoms involving an aberrant SSP-1 activity and/or an abnormal cell proliferation, differentiation, and/or survival, are, for example, antisense, ribozyme, and triple helix molecules described above. Examples of suitable compounds include the antagonists, agonists or homologues described in detail above.

Yet other SSP-1 therapeutics consist of a first peptide comprising a SSP-1 peptide capable of binding to a SSP-1 receptor, and a second peptide which is cytotoxic. Such therapeutics can be used to specifically target and lyse cells expressing or overexpressing a receptor for SSP-1.

Effective Dose

Toxicity and therapeutic efficacy of such compounds can be determined by standard pharmaceutical procedures in cell cultures or experimental animals, e.g., for determining The Ld₅₀ (The Dose Lethal To 50% Of The Population) And The Ed₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the oligomers of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

In clinical settings, the gene delivery systems for the therapeutic SSP-1 gene can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) PNAS 91: 3054-3057). A SSP-1 gene, such as any one of the sequences represented in the group consisting of SEQ ID NOS 1 and 3 or a sequence homologous thereto can be delivered in a gene therapy construct by electroporation using techniques described, for example, by Dev et al. ((1994) Cancer Treat Rev 20:105-115).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Diagnostic and Prognostic Assays

The present methods provides means for determining if a subject is at risk for developing a disorder characterized by an aberrant SSP-1 activity, such as aberrant cell proliferation, differentiation, and/or survival resulting for example in a neurodegenerative disease or cancer.

In one embodiment, the invention provides a method for determining whether a subject has genetic lesion in a SSP-1 gene. In another embodiment, the invention provides methods for determining whether a subject has an aberrant SSP-1 protein, resulting from aberrant post-translational modifications of the protein, such as aberrant phosphorylation or glycosylation. For example, a mutated SSP-1 protein can interact with a receptor other than an SSP- 1 receptor, or alternatively, a mutated S SP- 1 could interact with an SSP-1 receptor, but with an abnormal affinity. Binding of a mutated SSP-1 protein to an SSP-1 receptor could also result in an abnormal signal transduction.. Also, within the scope of the invention are methods for determining whether a subject has an aberrant expression level of a SSP-1 protein, which could be due to a genetic lesion in the SSP-1 gene.

In the diagnostic and prognostic assays described herein, in addition to the SSP-1 nucleic acid molecules and polypeptides described above, the present invention provides for the use of nucleic comprising at least a portion of a SSP-1 nucleic acid molecule, for example, at least a portion of a nucleic acid sequence shown in SEQ ID NOS: 1 or 3 or polypeptides as shown in SEQ ID No 2.

In preferred embodiments, the methods can be characterized as comprising detecting, in a sample of cells from the subject, the presence or absence of a genetic lesion characterized by at least one of (i) an alteration affecting the integrity of a gene encoding a SSP-1 protein, or (ii) the mis-expression of the SSP-1 gene. To illustrate, such genetic lesions can be detected by ascertaining the existence of at least one of (i) a deletion of one or more nucleotides from a SSP-1 gene, (ii) an addition of one or more nucleotides to a SSP-1 gene, (iii) a substitution of one or more nucleotides of a SSP-1 gene, (iv) a gross chromosomal rearrangement of a SSP-1 gene, (v) a gross alteration in the level of a messenger RNA transcript of a SSP-1 gene, (vii) aberrant modification of a SSP-1 gene, such as of the methylation pattern of the genomic DNA, (vii) the presence of a non-wild type splicing pattern of a messenger RNA transcript of a SSP-1 gene, (viii) a non-wild type level of a SSP-1 protein, (ix) allelic loss of a SSP-1 gene (x) inappropriate post-translational modification of a SSP-1 protein and (xi) errors and mutations in the promoter, which result in aberrant expression.

In an exemplary embodiment, there is provided a nucleic acid composition comprising a (purified) oligonucleotide probe including a region of nucleotide sequence which is capable of hybridizing to a sense or antisense sequence of a SSP-1 gene, such as represented by any of SEQ ID NOS: 1 or 3, or naturally occurring mutants thereof, or 5′ or 3′ flanking sequences or intronic sequences naturally associated with the subject SSP-1 genes or naturally occurring mutants thereof The nucleic acid of a cell is rendered accessible for hybridization, the probe is exposed to nucleic acid of the sample, and the hybridization of the probe to the sample nucleic acid is detected. Such techniques can be used to detect lesions at either the genomic or mRNA level, including deletions, substitutions, etc., as well as to determine mRNA transcript levels.

As set out above, one aspect of the present invention relates to diagnostic assays for determining, in the context of cells isolated from a patient, if mutations have arisen in one or more SSP-1 of the sample cells. In preferred embodiments, the method can be generally characterized as comprising detecting, in a sample of cells from the subject, the presence or absence of a genetic lesion characterized by an alteration affecting the integrity of a gene encoding a SSP-1. To illustrate, such genetic lesions can be detected by ascertaining the existence of at least one of (i) a deletion of one or more nucleotides from a SSP-1 gene, (ii) an addition of one or more nucleotides to a SSP-1 gene, (iii) a substitution of one or more nucleotides of a SSP-1 gene, and (iv) the presence of a non-wild type splicing pattern of a messenger RNA transcript of a SSP-1 gene. As set out below, the present invention provides a large number of assay techniques for detecting lesions in SSP-1 genes.

In certain embodiments, detection of the lesion comprises utilizing the probe/primer in a polymerase chain reacion (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the SSP-1 gene (see Abravaya et al. (1995) Nuc Acid Res 23:675-682). In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to a SSP-1 gene under conditions such that hybridization and amplification of the SSP-1 gene (if present) occurs, and (iv) detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Another embodiment of the invention provides for a nucleic acid composition comprising a (purified) oligonucleotide probe including a region of nucleotide sequence which is capable of hybridizing to a sense or antisense sequence of a SSP-1 gene, or naturally occurring mutants thereof, or 5′ or 3′ flanking sequences or intronic sequences naturally associated with the subject SSP-1 genes or naturally occurring mutants thereof. The nucleic acid of a cell is rendered accessible for hybridization, the probe is exposed to nucleic acid of the sample, and the hybridization of the probe to the sample nucleic acid is detected. Such techniques can be used to detect lesions at either the genomic or mRNA level, including deletions, substitutions, etc., as well as to determine mRNA transcript levels. Such oligonucleotide probes can be used for both predictive and therapeutic evaluation of allelic mutations which might be manifest in, for example, apoptosis or aberrant cell growth.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a SSP-1 gene.

Antibodies directed against wild type or mutant SSP-1 proteins, which are discussed, above, may also be used in disease diagnostics and prognostics. Such diagnostic methods, may be used to detect abnormalities in the level of SSP-1 protein expression, or abnormalities in the structure of an SSP-1 protein. Structural differences may include, for example, differences in the size, electronegativity, or antigenicity of the mutant SSP-1 protein relative to the normal SSP-1 protein. Protein from the tissue or cell type to be analyzed may easily be detected or isolated using techniques which are well known to one of skill in the art, including but not limited to western blot analysis. For a detailed explanation of methods for carrying out western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of SSP- 1 proteins. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the SSP-1 protein, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-SSP-1 protein specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alphaglycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, ο-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a cherniluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Moreover, it will be understood that any of the above methods for detecting alterations in a SSP-1 gene or gene product can be used to monitor the course of treatment or therapy.

Drug Screening Assays

The invention provides for SSP-1 therapeutic compounds for treating diseases or conditions caused by, or contributed to by an abnormal SSP-1 activity and for treating diseases characterized by an abnormal cell proliferation, differentiation, an/or survival. The compounds that can be used for this purpose can be any type of compound, including a protein, a peptide, peptidornimetic, small molecule, and nucleic acid. A nucleic acid can be, e.g., a gene, an antisense nucleic acid, a ribozyme, or a triplex molecule. A compound of the invention can be an agonist or an antagonist. A compound of the invention can be a compound which interacts with an SSP-1 protein to thereby modulate the interaction of the SSP-1 protein with a molecule, also referred to herein as “SSP-1 binding partner”, such as a receptor with which SSP-1 is capable of interacting. Preferred compounds which are capable of interacting with SSP-1 include anti-SSP-1 antibodies and derivatives thereof, as well as soluble forms of an SSP-1 binding partner, such as a soluble SSP-1 receptor. A preferred soluble SSP-1 receptor is an SSP-1 receptor fusion protein, e.g., SSP-1 receptor-immunoglobulin fusion protein (SSP-1 receptor-Ig protein). In other embodiments, the compound of the invention is a compound which is capable of interacting with an SSP-1 binding partner, such as an SSP-1 receptor, to thereby modulate the interaction of SSP-1 with the SSP-1 binding partner. For example, an SSP-1 therapeutic can be a dominant negative form of an SSP-1 protein, which is capable of binding to an SSP-1 receptor without transducing an intracellular signal and thereby prevents the natural or wild-type SSP-1 protein to interact with the receptor. In yet other embodiments, the compound of the invention is capable of binding to an SSP-1 receptor and mimic or agonize an SSP-1 protein. Preferred SSP-1 agonistic therapeutics include SSP-1 polypeptides or portions thereof which are capable of interacting with the receptor.

In yet other embodiments of the invention, an SSP.- 1 therapeutic is a compound which is capable of binding to an SSP-1 protein, e.g., a wild-type SSP-1 protein or a mutated form of an SSP-1 protein, and thereby degrades or causes the SSP-1 protein to be degraded. For example, such an SSP-1 therapeutic can be an antibody or derivative thereof which interacts specifically with an SSP-1 protein (either wild-type or mutated).

In addition, an SSP-1 therapeutic can be an SSP-1 polypeptide or other compound interacting with an SSP-1 receptor, which is linked to a cytotoxic molecule, to thereby lyse cells having SSP-1 receptors. In a preferred embodiment the SSP-1 therapeutic is an SSP-1 protein, e.g., a protein having the amino acid set forth in SEQ ID NO: 2 or a homolog thereof which is further linked to a cytotoxic molecule, e.g., a toxin.

In a further embodiment, the SSP-1 therapeutic of the invention is capable of acting on an SSP-1 gene, e.g., to modulate its expression.

The compounds of the invention can be identified using various assays depending on the type of compound and activity of the compound that is desired. Set forth below are at least some assays that can be used for identifying SSP-1 therapeutics. It is within the skill of the art to design additional assays for identifying SSP-1 therapeutics.

By making available purified and recombinant SSP-1 polypeptides, the present invention facilitates the development of assays which can be used to screen for drugs, including SSP-1 variants, which are either agonists or antagonists of the normal cellular function of the subject SSP-1 polypeptides, or of their role in the pathogenesis of cellular differentiation and/or proliferation and disorders related thereto. In one embodiment, the assay evaluates the ability of a compound to modulate binding between a SSP-1 polypeptide and a molecule, e.g., an SSP-1 receptor. A variety of assay formats will suffice and, in light of the present inventions, will be comprehended by a skilled artisan.

Cell-Free Assays

Cell-free assays can be used to identify compounds which interact with a SSP-1 protein or SSP-1 receptor. Such assays are available for testing compounds which are proteins, e.g., soluble SSP-1 receptor proteins or variants thereof, as well as for testing compounds which are peptidomimetics, small molecules or nucleic acids. The specific assay used for testing these compounds may vary with the type of compound.

In one embodiment, a compound that interacts with a SSP-1 protein or an SSP-1 receptor is identified by screening, e.g., a library of compounds, for binding to a recombinant or purified SSP-1 protein or an SSP-1 receptor or at least a portion of either of these proteins. Such assays can involve labeling one or the two components and measuring the extent of their interaction, by, e.g.,determining the level of the one or two labels. In these assays, it may be preferable to attach the SSP-1 protein or the SSP-1 receptor to a solid phase surface. Methods for achieving this are further described infra.

In one embodiment, the library of compounds is a library of small molecules. In another embodiment, the library of compounds is a library of SSP-1 protein or SSP-1 receptor variants, which can be produced according to methods described infra.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with upstream or downstream elements. Accordingly, in an exemplary screening assay of the present invention, the compound of interest is contacted with an SSP-1 protein or an SSP-1 binding partner, e.g., a receptor. The receptor can be soluble or the receptor can be present on a cell surface. To the mixture of the compound and the SSP-1 protein or SSP-1 binding partner is then added a composition containing an SSP-1 binding partner or an SSP-1 protein, respectively. Detection and quantification of complexes of SSP-1 proteins and SSP-1 binding partners provide a means for determining a compound's efficacy at inhibiting (or potentiating) complex formation between SSP-1 and a binding partner. The efficacy of the compound can be assessed by generating dose response curves from data obtained using various concentrations of the test compound. Moreover, a control assay can also be performed to provide a baseline for comparison. In the control assay, isolated and purified SSP-1 polypeptide or binding partner is added to a composition containing the SSP-1 binding partner or SSP-1 polypeptide, and the formation of a complex is quantitated in the absence of the test compound.

Complex formation between an SSP-1 protein and an SSP-1 binding partner may be detected by a variety of techniques. Modulation of the formation of complexes can be quantitated using, for example, detectably labeled proteins such as radiolabeled, fluorescently labeled, or enzymatically labeled SSP-1 proteins or SSP-1 binding partners, by immunoassay, or by chromatographic detection.

Typically, it will be desirable to immobilize either SSP-1 or its binding partner to facilitate separation of complexes from uncomplexed forms of one or both of the proteins, as well as to accommodate automation of the assay. Binding of SSP-1 to an SSP-1 binding partner, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/SSP-1 (GST/SSP-1) fusion proteins can be adsorbed onto glutathione sepharose beads (Sigma Chemical, St. Louis, Mo.) or glutathione derivatized microtitre plates, which are then combined with the cell lysates, e.g. an ³⁵S-labeled, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of SSP-1 protein or SSP-1 binding partner found in the bead fraction quantitated from the gel using standard electrophoretic techniques such as described in the appended examples.

Other techniques for immobilizing proteins on matrices are also available for use in the subject assay. For instance, either SSP-1 or its cognate binding partner can be immobilized utilizing conjugation of biotin and streptavidin. For instance, biotinylated SSP-1 molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with SSP-1 can be derivatized to the wells of the plate, and SSP-1 trapped in the wells by antibody conjugation. As above, preparations of a SSP-1 binding protein and a test compound are incubated in the SSP-1 presenting wells of the plate, and the amount of complex trapped in the well can be quantitated. Exemplary methods for detecting such complexes, in addition to those described above for the GST-immobilized complexes, include immunodetection of complexes using antibodies reactive with the SSP-1 binding partner, or which are reactive with SSP-1 protein and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion protein with the SSP-1 binding partner. To illustrate, the SSP-1 binding partner can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as anti-SSP-1 antibodies, can be used. Alternatively, the protein to be detected in the complex can be “epitope tagged” in the form of a fusion protein which includes, in addition to the SSP-1 sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharamacia, N.J.).

Cell Based Assays

In addition to cell-free assays, such as described above, the readily available source of SSP-1 proteins provided by the present invention also facilitates the generation of cell-based assays for identifying small molecule agonists/antagonists and the like. For example, cells expressing an SSP-1 binding partner, e.g., an SSP-1 receptor protein, are incubated in the presence or absence of a test agent of interest with or without an SSP-1 protein, and the modulation and the modulation of an SSP-1 activity is measured. As with the cell-free assays, agents which produce a statistically significant change in an SSP-1 activity (either inhibition or potentiation) can be identified. In an illustrative embodiment, the expression or activity of a SSP-1 is modulated in cells and the effects of compounds of interest on the readout of interest (such as tissue differentiation, proliferation, tumorigenesis) are measured. For example, the expression of genes which are up- or down-regulated in response to a SSP-1 dependent signal cascade can be assayed. In preferred embodiments, the regulatory regions of such genes, e.g., the 5′ flanking promoter and enhancer regions, are operably linked to a detectable marker (such as luciferase) which encodes a gene product that can be readily detected.

In one embodiment, a test compound that modifies an SSP-1 activity can be identified by incubating a cell having an SSP-1 receptor protein with the test compound and measuring signal transduction from the SSP-1 receptor protein. Comparison of the signal transduction in the cells incubated with or without the test compound will reveal whether the test compound is an SSP-1 therapeutic.

In another embodiment, a silicon-based device, called a microphysiometer, can be used to detect and measure the response of cells having an SSP-1 receptor protein to test compounds to identify SSP-1 therapeutics. This instrument measures the rate at which cells acidify their environment, which is indicative of cellular growth and/or differentiation (McConnel et al. (1992) Science 257:1906).

Transgenic animals

Transgenic animals can be used, e.g., to identify SSP-1 therapeutics. One aspect of the present invention concerns transgenic animals which are comprised of cells (of that animal) which contain a transgene of the present invention and which preferably (though optionally) express an exogenous SSP-1 protein in one or more cells in the animal. A SSP-1 transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a SSP-1 protein can be essential for many forms of lineage analysis and can additionally provide a means to assess the effects of, for example, lack of SSP-1 expression which might grossly alter development in small patches of tissue within an otherwise normal embryo. Toward this and, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques which allow for the expression of transgenes can be regulated via site-specific genetic manipulation in vivo are known to those skilled in the art. For instance, genetic systems are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject SSP-1 proteins. For example, excision of a target sequence which interferes with the expression of a recombinant SSP-1 gene, such as one which encodes an antagonistic homolog or an antisense transcript, can be designed to activate expression of that gene. This interference with expression of the protein can result from a variety of mechanisms, such as spatial separation of the SSP-1 gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxp sequences are 34 base pair nucleotide repeat sequences to which the Cre recombinase binds and are required for Cre recombinase mediated genetic recombination. The orientation of loxp sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxp sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant SSP-1 protein can be regulated via control of recombinase expression.

Use of the cre/loxP recombinase system to regulate expression of a recombinant SSP-1 protein requires the construction of a transgenic animal containing transgenes encoding both the Cre recombinase and the subject protein. Animals containing both the Cre recombinase and a recombinant SSP-1 gene can be provided through the construction of “double” transgenic animals. A convenient method for providing such animals is to mate two transgenic animals each containing a transgene, e.g., a SSP-1 gene and recombinase gene.

One advantage derived from initially constructing transgenic animals containing a SSP-1 transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic SSP-1 transgene is silent will allow the study of progeny from that founder in which disruption of SSP-1 mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the SSP-1 transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g., a recombinase or a prokaryotic protein, is delivered to the tissue and caused to be expressed, such as in a cell-type specific manner. By this method, a SSP-1 transgene could remain silent into adulthood until “turned on” by the introduction of the transactivator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H—2^{b}, H—2^{d} or H—2^{q} haplotypes such as C57BL/6 or DBA/1. The line(s) used to practice this invention may themselves be transgenics, and/or may be knockouts (i.e., obtained from animals which have one or more genes partially or completely suppressed) In one embodiment, the transgene construct is introduced into a single stage embryo. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of a SSP-1 protein (either agonistic or antagonistic), and antisense transcript, or a SSP-1 mutant. Further, in such embodiments the sequence will be attached to a transcriptional control element, e.g., a promoter, which preferably allows the expression of the transgene product in a specific type of cell.

Retroviral infection can also be used to introduce transgene into a nonhuman animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipula SSP-1ng the Mouse Embryo, Hogan eds. (Cold Spring Harbor Laboratory Press, Cold Spring Harbor, 1986). The viral vector system used to introduce the transgene is typically a replicationdefective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

In one embodiment, gene targeting, which is a method of using homologous recombination to modify an animal's genome, can be used to introduce changes into cultured embryonic stem cells. By targeting a SSP-1 gene of interest in ES cells, these changes can be introduced into the germlines of animals to generate chimeras. The gene targeting procedure is accomplished by introducing into tissue culture cells a DNA targeting construct that includes a segment homologous to a target SSP-1 locus, and which also includes an intended sequence modification to the SSP-1 genomic sequence (e.g., insertion, deletion, point mutation). The treated cells are then screened for accurate targeting to identify and isolate those which have been properly targeted.

Gene targeting in embryonic stem cells is in fact a scheme contemplated by the present invention as a means for disrupting a SSP-1 gene function through the use of a targeting transgene construct designed to undergo homologous recombination with one or more SSP-1 genomic sequences. The targeting construct can be arranged so that, upon recombination with an element of a SSP-1 gene, a positive selection marker is inserted into (or replaces) coding sequences of the targeted signalling gene. The inserted sequence functionally disrupts the SSP-1 gene, while also providing a positive selection trait. Exemplary SSP-1 targeting constructs are described in more detail below.

Generally, the embryonic stem cells (ES cells) used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al. (1985) J. Embryol. Exp. Morphol. 87:27-45). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934) Still another preferred ES cell line is the WW6 cell line (ioffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. [1986]).

Insertion of the knockout construct into the ES cells can be accomplished using a variety of methods well known in the art including for example, electroporation, microinjection, and calcium phosphate treatment. A preferred method of insertion is electroporation.

Each knockout construct to be inserted into the cell must first be in the linear form. Therefore, if the knockout construct has been inserted into a vector (described infra), linearization is accomplished by digesting the DNA with a suitable restriction endonuclease selected to cut only within the vector sequence and not within the knockout construct sequence.

For insertion, the knockout construct is added to the ES cells under appropriate conditions for the insertion method chosen, as is known to the skilled artisan. Where more than one construct is to be introduced into the ES cell, each knockout construct can be introduced simultaneously or one at a time.

If the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knockout construct .

Screening can be accomplished using a variety of methods. Where the marker gene is an antibiotic resistance gene, for example, the ES cells may be cultured in the presence of an otherwise lethal concentration of antibiotic. Those ES cells that survive have presumably integrated the knockout construct. If the marker gene is other than an antibiotic resistance gene, a Southern blot of the ES cell genomic DNA can be probed with a sequence of DNA designed to hybridize only to the marker sequence Alternatively, PCR can be used. Finally, if the marker gene is a gene that encodes an enzyme whose activity can be detected (e.g., β-galactosidase), the enzyme substrate can be added to the cells under suitable conditions, and the enzymatic activity can be analyzed. One skilled in the art will be familiar with other useful markers and the means for detecting their presence in a given cell. All such markers are contemplated as being included within the scope of the teaching of this invention.

The knockout construct may integrate into several locations in the ES cell genome, and may integrate into a different location in each ES cell's genome due to the occurrence of random insertion events. The desired location of insertion is in a complementary position to the DNA sequence to be knocked out, e.g., the SSP-1 coding sequence, transcriptional regulatory sequence, etc. Typically, less than about 1-5% of the ES cells that take up the knockout construct will actually integrate the knockout construct in the desired location. To identify those ES cells with proper integration of the knockout construct, total DNA can be extracted from the ES cells using standard methods. The DNA can then be probed on a Southern blot with a probe or probes designed to hybridize in a specific pattern to genomic DNA digested with particular restriction enzyme(s). Alternatively, or additionally, the genomic DNA can be amplified by PCR with probes specifically designed to amplify DNA fragments of a particular size and sequence (i.e., only those cells containing the knockout construct in the proper position will generate DNA fragments of the proper size).

After suitable ES cells containing the knockout construct in the proper location have been identified, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the knockout construct into the developing embryo. For instance, as the appended Examples describe, the transformed ES cells can be microinjected into blastocytes.

The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan, and are set forth by, e.g., Bradley et al. (supra).

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the SSP-1 gene knocked out in various tissues of the offspring by probing the Western blot with an antibody against the particular SSP-1 protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g., by homologous recombination to insert target sequences, such that tissue specific and/or temporal control of inactivation of a SSP-1 gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s) .

Additional Uses for SSP-1 Proteins and Nucleic Acids

In another embodiment, the SSP-1 proteins of the invention can be used as tissue culture additives for culture of cell lines, tissues, and primary cells, i.e., cells obtained freshly from a subject. Accordingly, an SSP-1-protein or variant thereof, such as a recombinant form of an SSP-1 protein is added in amounts sufficient to modulate cell growth, differentiation or survival of the cells. It may also be preferable to use forms of SSP-1 which have been modified to increase its half-life.

Based on the fact that SSP-1 is a secreted protein, it is possible that SSP-1 is a protein capable of binding other extracellular molecules. Accordingly, SSP-1 could have a detoxifying role or could function as a carrier protein for specific molecules. For example, SSP-1 could bind to a molecule and thereby eliminate the molecule from the extracellular medium. The molecule can be a metal, e.g., heavy metal. The molecule can also be a protein, a lipid, a glycoprotein or any derivative of such molecules. SSP-1 can also have a structural role in the extracellular matrix, such as a role of “organizing” the extracellular matrix, such as osteocalcin which is capable of binding calcium phosphate in bone matrix. SSP-1 can also be an extracellular enzyme, e.g., digestive enzyme, or an enzyme capable of degrading extracellular matrix proteins.

The SSP-1 nucleic acids of the invention can further be used in the following assays. In one embodiment, the human SSP-1 nucleic acid having SEQ ID NO: 1 or a portion thereof, or a nucleic acid which hybridizes thereto can be used to determine the chromosomal localization of an SSP-1 gene. Comparison of the chromosomal location of the SSP-1 gene with the location of chromosomal regions which have been shown to be associated with specific diseases or conditions, e.g., by linkage analysis (coinheritance of physically adjacent genes), can be indicative of diseases or conditions in which SSP-1 may play a role. A list of chromosomal regions which have been linked to specific diseases can be found, for example, in V. McKusick, Mendelian Inheritance in Man (available on line through Johns Hopkins University Welch Medical Library). Furthermore, the SSP-1 gene can also be used as a chromosomal marker in genetic linkage studies involving genes other than SSP-1.

Chromosomal localization of a gene can be performed by several methods well known in the art. For example, Southern blot hybridization or PCR mapping of somatic cell hybrids can be used for determining on which chromosome or chromosome fragment a specific gene is located. Other mapping strategies that can similarly be used to localize a gene to a chromosome or chromosomal region include in situ hybridization, prescreening with labeled flow-sorted chromosomes and preselection by hybridization to construct chromosome specific-cDNA libraries.

Furthermore, fluorescence in situ hybridization (FISH) of a nucleic acid, e.g., an SSP-1 nucleic acid, to a metaphase chromosomal spread is a one step method that provides a precise chromosomal location of the nucleic acid. This technique can be used with nucleic acids as short as 500 or 600 bases; however, clones larger than 2,000 bp have a higher likelihood of binding to a unique chromosomal location with sufficient signal intensity for simple detection. Such techniques are described, e.g, in Verma et al., Human Chromosomes: a Manual of Basic Techniques, Pergamon Press, New York (1988). Using such techniques, a gene can be localized to a chromosomal region containing from about 50 to about 500 genes.

If the SSP-1 gene is shown to be localized in a chromosomal region which cosegregates, i.e., which is associated, with a specific disease, the differences in the cDNA or genomic sequence between affected and unaffected individuals are determined. The presence of a mutation in some or all of the affected individuals but not in any normal individuals, will be indicative that the mutation is likely to be causing or contributing to the disease.

The present invention is further illustrated by the following examples which should not be construed as limiting in any way. The contents of all cited references (including literature references, issued patents, published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, ₂nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization(B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

Cloning and Analysis of Human SSP-1

A full length cDNA encoding human SSP-1 protein having SEQ ID NO: 2 has been isolated by signal sequence trap assay from a library prepared from human fetal brain. Briefly, a randomly primed cDNA library using mRNA prepared from human fetal brain tissue (Clontech, Palo Alto Calif.) was made by using the Statagene-ZAP-cDNA Synthesis kit, (catalog #20041). the cDNA was ligated into the mammalian expression vector pTrap adjacent to a cDNA encoding placental alkaline phosphatase lacking a secretory signal. The plasmids were transformed into E. Coli and DNA was prepared using the Wizard DNA purification kit (Promega). DNA was transfected COS-7 cells with lipofectamine (Gibco-BRL). After 48 hours incubation the COS cell supernatants were assayed for alkaline phosphatase on a Wallace Micro-Beta scintillation counter using the Phosph-Light kit (Tropix Inc. Catalog #BP300). The individual plasmid DNAs scoring positive in the Cos cell Alkaline secretion assay were further analyzed by DNA sequencing using standard procedures.

The nucleic acid sequence encoding the full length SSP-1 protein is shown in FIG. 1 and is set forth in SEQ ID NO: 1. The full length protein encoded by this nucleic acid has 172 amino acids and has the amino acid sequence shown in FIG. 1 and set forth is SEQ ID NO: 2. The coding portion of SEQ ID NO: 1 is set forth in SEQ ID NO: 3 and corresponds to the sequence from nucleotide 591 to 1106 of SEQ ID NO. 1.

An amino acid sequence analysis indicates that the SSP-1 protein comprises an N-terminal hydrophobic domain, as shown in FIG. 2, and contains a signal peptide of about 23 amino acids. Accordingly, the SSP-1 protein contains a signal peptide from amino acid 1 to about amino acid 23 of SEQ ID NO: 2. In addition, the fact that SSP-1 has been cloned by using a signal sequence trap system further indicates that SSP-1 is a secreted protein.

A blast search of the nucleic acid and the amino acid sequences of SSP- 1 has revealed that SSP-1 has significant homology to two proteins, which are homologous to each other, and which are expressed during the development of the cement gland in Xenopus. One protein is a 183 amino acid long protein termed XAG having GenBank Accession No. U76752 (Sive et al. (1989) Cell 58:171-180; Sive and Bradley (1996) Dev. Dyn. 205:265). The second protein is a 173 amino acid long protein termed np77 having GenBank Accession No. U82110. An alignment of the amino acid sequences of the two Xenopus cement gland proteins and human SSP-1 having SEQ ID NO: 2 is shown in FIG. 3. This sequence alignment shows an overall homology between the three proteins. More specifically, the amino acid sequence of the human SSP-1 having SEQ ID NO: 2 is about 29.7% identical to the amino acid sequence of XAG. The amino acid sequence of human SSP-1 having SEQ ID NO: 2 is about 29.4% identical to the amino acid sequence np77. Furthermore, specific amino acid sequences are present in the three sequences, thus indicating that these sequences probably encode specific domains.

Expression of Recombinant SSP-1 in COS Cells

This example describes a method for producing recombinant full length human SSP-1 in a mammalian expression system.

An expression construct containing a nucleic acid encoding a full length human SSP-1 protein can be constructed as follows. A nucleic acid encoding the full length human SSP-1 protein is obtained by reverse transcription (RT-) PCR of mRNA extracted from human cells expressing SSP-1, e.g., human fetal brain cells using PCR primers based on the sequence set forth in SEQ ID NO: 1. The PCR primers further contain appropriate restriction sites for introduction into the expression plasmid. The amplified nucleic acid is then inserted in a eukaryotic expression plasmid such as pcDNAI/Amp (Invitrogen) containing: 1) SV40 origin of replication, 2) ampicillin resistance gens, 3) E. coli replication origin, 4) CMV promoter followed by a polylinker region, a SV40 intron and polyadenylation site. A DNA fragment encoding the full length human SSP-1 and a HA or myc tag fused in frame to its 3′ end is then cloned into the polylinker region of the. The HA tag corresponds to an epitope derived from the influenza hemagglutinin protein as previously described (I. Wilson, H. Niman, R. Heighten, A Cherenson, M. Connolly, and R. Lerner, 1984, Cell 37, 767). The infusion of HA tag to SSP-1 allows easy detection of the recombinant protein with an antibody that recognizes the HA epitope.

For expression of the recombinant SSP-1, COS cells are transfected with the expression vector by DEAE-DEXTRAN method. (J. Sambrook, E. Fritsch, T. Maniatis, Molecular Cloning: A Laboratory Manual, Cold Spring Laboratory Press, (1989)). The expression of the SSP-1 -HA protein can be detected by radiolabelling and immunoprecipitation. (E. Harlow, D. Lane, Antibodies: A Laboratory Manual, Cold Spring Harbor Laboratory Press, (1988)). For this, transfected cells are labelled with ³⁵S-cysteine two days post transfection. Culture media is then collected and the SSP-1 protein immunoprecipitated with an HA specific monoclonal antibody. Proteins precipitated can then be analyzed on SDS-PAGE gels.

If a membrane form of an SSP-1 protein is expressed in the cells, the transfected cells can be lysed with detergent (RIPA buffer (150 mM NaCl 1% NP-40, 0.1% SDS, 1% NP-40, 0.5% DOC, 50 mM Tris, pH 7.5). (Wilson, I. et al., Id. 37:767 (1984)). Cell lysates are then precipitated with an HA specific monoclonal and analyzed on SDS-PAGE gel.

A similar method can be used to prepare large amounts of secreted SSP-1 protein. In a preferred embodiment, cells are stably transfected with an expression vector encoding an SSP-1 protein or variant thereof, such as the expression vector described above and the recombinant SSP-1 protein is collected from the supernatant of the culture.

Equivalents

Those skilled in the art will recognize, or be able to ascertain using no more than routine experimentation, many equivalents of the specific embodiments of the invention described herein. Such equivalents are intended to be encompassed by the following claims.

1. An isolated nucleic acid comprising a nucleotide sequence encoding an SSP-1 polypeptide.

2. The isolated nucleic acid of claim 1, wherein the SSP-1 polypeptide is a mammalian polypeptide.

3. The isolated nucleic acid of claim 2, wherein the SSP-1 polypeptide is a human polypeptide.

4. The isolated nucleic acid of claim 1, wherein the SSP- I polypeptide has an overall amino acid homology of at least about 70% with the amino acid sequence set forth in SEQ ID NO: 2.

5. The isolated nucleic acid of claim 4, wherein the SSP-1 polypeptide has the amino acid sequence set forth in SEQ ID NO: 2.

6. The isolated nucleic acid of claim 1, wherein the SSP-1 polypeptide is a mature form of an SSP-1 polypeptide.

7. The isolated nucleic acid of claim 6, wherein the SSP-1 polypeptide comprises at least about amino acid 21 to about amino acid 172 of SEQ ID NO: 2.

8. The isolated nucleic acid of claim 1, wherein the SSP-1 polypeptide comprises an amino acid sequence which is at least about 70% similar to at least about 15 consecutive amino acid residues of SEQ ID NO: 2.

9. The nucleic acid of claim 1, wherein the SSP-1 polypeptide comprises an amino acid sequence which is at least about 70% similar to at least about 50 consecutive amino acid residues of SEQ ID NO: 2.

10. The isolated nucleic acid of claim 1, wherein the SSP-1 polypeptide comprises at least about 10 consecutive amino acids of SEQ ID NO: 2.

11. The isolated nucleic acid of claim 1, wherein the SSP-1 polypeptide comprises at least about 50 consecutive amino acids of SEQ ID NO: 2.

12. The isolated nucleic acid of claim 1, wherein the SSP-1 polypeptide is a functional SSP-1 polypeptide.

13. The isolated nucleic acid of claim 1, wherein the SSP-1 polypeptide is capable of interacting with a molecule.

14. The isolated nucleic acid of claim 13, wherein the molecule is a receptor.

15. The isolated nucleic acid of claim 1, which has an overall nucleotide identity of at least about 70% with the nucleotide sequence set forth in SEQ ID NO: 1.

16. The isolated nucleic acid of claim 1, which has an overall nucleotide identity of at least about 70% with the nucleotide sequence set forth in SEQ ID NO: 3.

17. An isolated nucleic acid comprising a nucleotide sequence which is at least about 80% identical to at least about 50 consecutive nucleotides SEQ ID NO: 1 or complement thereof.

18. An isolated nucleic acid comprising a nucleotide sequence of at least about 20 consecutive nucleotides of SEQ ID NO: 1 or 3 or complement thereof.

19. The isolated nucleic acid of claim 18, further comprising a label.

20. The isolated nucleic acid of claim 1, which hybridizes under high stringency hybridization conditions to the nucleic acid set forth in SEQ ID NO: 1 or to the complement thereof.

21. A vector comprising a nucleic acid of claim 1.

22. A host cell comprising the vector of claim 21.

23. An isolated SSP-1 polypeptide.

24. The isolated SSP-1 polypeptide of claim 23, wherein the SSP-1 polypeptide is a mammalian polypeptide.

25. The isolated SSP-1 polypeptide of claim 24, wherein the SSP-1 polypeptide is a human polypeptide.

26. The isolated SSP-1 polypeptide of claim 23, wherein the SSP-1 polypeptide has an overall amino acid homology of at least about 70% with the amino acid sequence set forth in SEQ ID NO: 2.

27. The SSP-1 polypeptide of claim 26, wherein the SSP-1 polypeptide has the amino acid sequence set forth in SEQ ID NO: 2.

28. The isolated SSP-1 polypeptide of claim 23, wherein the SSP-1 polypeptide is a mature form of an SSP-1 polypeptide.

29. The isolated SSP-1 polypeptide of claim 2 8, wherein the SSP-1 polypeptide comprises at least about amino acid 21 to about amino acid 172 of SEQ ID NO: 2.

30. The isolated SSP-1 polypeptide of claim 23, wherein the SSP-1 polypeptide comprises an amino acid sequence which is at least about 70% similar to at least about 15 consecutive amino acid residues of SEQ ID NO: 2.

31. The SSP-1 polypeptide of claim 23, wherein the SSP-1 polypeptide comprises an amino acid sequence which is at least about 70% similar to at least about 50 consecutive amino acid residues of SEQ ID NO: 2.

32. The isolated SSP-1 polypeptide of claim 23, wherein the SSP-1 polypeptide comprises at least about 10 consecutive amino acids of SEQ ID NO: 2.

33. The isolated SSP-1 polypeptide of claim 23, wherein the SSP-1 polypeptide comprises at least about 50 consecutive amino acids of SEQ ID NO: 2.

34. The isolated SSP-1 polypeptide of claim 23, wherein the SSP-1 polypeptide is a functional SSP-1 polypeptide.

35. The isolated SSP-1 polypeptide of claim 23, wherein the SSP-1 polypeptide is capable of interacting with a molecule.

36. The isolated nucleic acid of claim 35, wherein the molecule is a receptor.

37. A method for modulating an SSP-1 activity, comprising contacting an SSP-1 polypeptide with a compound which is capable of modulating a SSP-1 activity, such that the SSP-1 activity is modulated.

38. The method of claim 37, wherein the SSP-1 activity is modulation of cell proliferation, differentiation or cell survival.

39. The method of claim 38, wherein the compound is an antagonist.

40. The method of claim 39, wherein the antagonist inhibits the interaction of SSP-1 with an SSP-1 receptor.

41. The method of claim 37, wherein the SSP-1 activity is binding to a molecule.

42. The method of claim 41, wherein the molecule is an SSP-1 receptor.

43. A method for modulating growth, differentiation, or survival of a cell, comprising contacting the cell with an SSP-1 compound which is capable of modulating cell growth, differentiation, or survival.

44. The method of claim 43, wherein the compound is an agonist of a SSP-1 activity.

45. The method of claim 44, wherein the compound is an antagonist of a SSP-1 activity.

46. The method of claim 45, wherein the compound is selected from the group consisting of a polypeptide, a nucleic acid, a peptidomimetic, and a small molecule.

47. The method of claim 46, wherein the nucleic acid is selected from the group consisting of a gene replacement, an antisense, a ribozyme, and a triplex nucleic acid. The method of claim 43, wherein the compound interacts with an SSP-1 protein.

48. The method of claim 43, wherein the compound interacts with an SSP-1 receptor.

49. The method of claim 48, wherein the compound is an SSP-1 polypeptide.

50. A method for treating or preventing a disease caused by or contributed to by an aberrant SSP-1 activity in a subject, comprising administering to the subject an effective amount of a pharmaceutical composition comprising a compound which is capable of modulating a SSP-1 activity, such that the disease is treated or prevented in the subject.

51. The method of claim 50, wherein the disease is a hyper- or hypoproliferative disease.

52. The method of claim 52, wherein the compound is an SSP-1 polypeptide.

53. A method for treating or preventing a disease associated with an abnormal cell proliferation, differentiation or survival in a subject, comprising administering to the subject a pharmaceutically effective amount of an SSP-1 therapeutic.

54. The method of claim 53, wherein the disease is a hyperproliferative disease.

55. The method of claim 53, wherein the disease is a hypoproliferative disease.

56. A method for identifying a SSP-1 therapeutic, comprising
(i) combining a SSP-1 protein, a SSP-1 binding partner, and a test compound under conditions wherein, but for the test compound, the SSP-1 protein and SSP-1 binding partner are able to interact; and
(ii) detecting the formation of a SSP-1 protein/SSP-1 binding partner complex, such that a difference in the formation of a SSP-1 protein/SSP-1 binding partner complex in the presence of a test compound relative to the absence of the test compound is indicative that the test compound is a SSP-1 therapeutic.

57. The method of claim 56, wherein the SSP-1 binding partner is an SSP-1 receptor.

58. A method for determining whether a subject is at risk of developing a disease or condition which is caused or contributed to by an aberrant SSP-1 activity, comprising measuring in the subject or in a sample obtained from the subject at least one SSP-1 activity, wherein a difference in the SSP-1 activity relative to the SSP-1 activity in a normal subject indicates that the subject is at risk of developing a disease caused by or contributed to by an aberrant SSP-1 activity.

59. The method of claim 58, wherein a SSP-1 activity is determined by measuring the protein level of an SSP-1 protein.

60. The method of claim 58, comprising the step of determining whether the SSP-1 gene of the subject comprises a genetic lesion.

61. The method of claim 60, wherein the step of determining whether the SSP-1 gene comprises a genetic lesion, further comprises the steps of:
(i) contacting a nucleic acid comprising at least a portion of the SSP-1 gene from a subject with at least one nucleic acid probe capable of interacting with a wild-type SSP-1 gene; and
(ii) detecting the formation of a hybrid between the portion of the SSP-1 gene from the subject and the at least one nucleic acid probe, such that the extent of formation of a hybrid between the portion of the SSP-1 gene from the subject and the at least one nucleic acid indicates whether the subject is at risk of developing a disease associated with an aberrant SSP-1 activity.
